(19) United States
(12) Patent Application Publication
(10) Pub. No.: US 2025/0380904 A1
(43) Pub. Date: Dec. 18, 2025

(54) Fluid Monitoring System

(71) Applicant: The University Court of the University of Edinburgh, Edinburgh (GB)

(72) Inventors: Tughrul Sati Arslan, Edinburgh (GB); Imran Mohammed Saied, Edinburgh (GB)

(21) Appl. No.: 18/875,528

(22) PCT Filed: Jun. 15, 2023
(86) PCT No.: PCT/GB2023/051577

§ 371 Date: Dec. 16, 2024

(30) Foreign Application Priority Data
Jun. 16, 2022 (GB) 2208889.2
Jan. 11, 2023 (GB) 2300421.1

(51) Int. Cl. A61B 5/00; A61B 5/05

(52) U.S. Cl. CPC A61B 5/4875; A61B 5/05; A61B 5/6891; A61B 5/6893; A61B 5/7271; A61B 5/746; A61B 2503/04; A61B 2503/12; A61B 2560/0462; A61B 2562/043; A61B 2562/16

(57) ABSTRACT

The present disclosure relates to a fluid level and/or fluid composition monitoring system including a radiofrequency sensor configured to be included in an item of furnishing or other item for use by a patient or other subject and a control system configured to receive a signal from the radiofrequency sensor and to determine a level of fluid and/or a composition of fluid in a part of a patient or other subject's body in vivo based on the signal.

### FIELD

The present disclosure relates to a fluid level and/or fluid composition monitoring system, an item of furnishing or other item and associated methods.

### BACKGROUND

Some patients of care homes and hospitals may spend long periods of time sitting or lying down. Similarly, other subjects or persons may spend long periods of time sitting or lying down in their home. A sudden change or urge in their bodies may cause them to get up suddenly, which in turn may lead to exertion on their muscles and/or falls, causing injuries. The sudden change or urge in the patient or other subject's body may be due to an increase in fluid in one or more organs of the patient. For example, an increase in the fluid in the stomach, kidneys and liver may not only cause a frequent need to use the bathroom, but the weight of the fluid in the organs may affect the patient or other subject's motion. This may lead to the exertion of muscles and/or falls. Additionally, when the patient or other subject is lying or sitting down, it may be difficult for them to realise how much fluid there is in their body and/or whether or not they need to use the bathroom.

This background serves only to set a scene to allow a person skilled in the art to better appreciate the following description. Therefore, none of the above discussion should necessarily be taken as an acknowledgement that that discussion is part of the state of the art or is common general knowledge. One or more aspects/embodiments of the disclosure may or may not address one or more of the background issues.

### SUMMARY

According to a first aspect of the present disclosure there is provided a fluid level and/or fluid composition monitoring system comprising a radiofrequency sensor configured to be included in an item of furnishing or other items for use by a patient or other subject and a control system configured to receive a signal from the radiofrequency sensor and to determine a level of fluid and/or a composition of fluid in a part of a patient or other subject's body in vivo based on the signal.

The term “fluid” may be considered as encompassing a single fluid, a mixture or combination of at least two fluids, e.g. at least two different fluids, or a mixture or combination of a fluid with a substance.

The system may allow for the prediction of one or more possible oncoming adverse events, such as a fall, incontinence, dehydration, a disease or other medical condition. For example, a determined level of fluid and/or composition of fluid may be indicative of the one or more possible oncoming adverse events. The determined level of fluid and/or composition of fluid may be used by a doctor, another health professional or another person to make a diagnosis of the disease or medical condition and/or to assist a patient or other subject with their need. As such, the system may allow for a level of hydration and/or a state of health of the patient or other subject to be monitored.

By configuring the radiofrequency sensor to be included in the item of furnishing or other item, a level of fluid and/or a composition of fluid in a part of the patient or other subject's body may be unobtrusively and/or noninvasively monitored in vivo. This may also allow the system to be used in any care environment, medical environment, home environment or any other environment.

The radiofrequency sensor may be configured to be embedded or integrated in the item of furnishing or other item.

The signal may be or comprise a reflected signal. The signal may comprise a transmitted signal. For example, the signal may be or comprise a portion of another signal that has been reflected by the part of the patient or other subject's body. The signal may be or comprise a portion of another signal that has been transmitted through the part of the patient or other subject's body.

The control system may be configured to determine the level of fluid and/or the composition of fluid in the part of the patient or other subject's body in vivo based on the other signal. The radiofrequency sensor may be configured to transmit the other signal. Alternatively, the other signal may be transmitted by another radiofrequency sensor.

The radiofrequency sensor may be configured to transmit a first signal to the part of the patient or other subject's body. The radiofrequency sensor may be configured to receive a second signal from the part of the patient or other subject's body. The second signal may comprise a portion of the first signal that has been reflected by the part of the patient or other subject's body. The signal may be or comprise the second signal. The other signal may be or comprise the first signal.

The system may be configured to continuously monitor the level of fluid and/or the composition of fluid in the part of the patient or other subject's body. For example, the radiofrequency sensor may be configured to alternately transmit the first signal to the part of the patient or other subject's body and receive the second signal from the part of the patient or other subject's body. The system may be configured to monitor the level of fluid and/or the composition of fluid at selected time intervals, such as every 10 minutes, every 30 minutes, every 1 hour, 2, 3, 4, 5 or 10 hours. For example, the radiofrequency sensor may be configured to transmit the first signal to the part of the patient or other subject's body and to receive the second signal from the part of the patient or other subject's body at the selected time interval. The system may be configured to monitor the level of fluid and/or the composition of fluid in the part of the patient or other subject's body for a duration. For example, the radiofrequency sensor may be configured to alternately transmit the first signal to the part of the patient or other subject's body and receive the second signal from the part of the patient or other subject's body during the duration. The duration may be in the range of about 5 minutes to 60 minutes, 10 minutes to 120 minutes, 1 hour to 24 hours, 12 hours to 72 hours or the like. The system may be configured to monitor the level of fluid and/or the composition of fluid at one or more intervals, e.g. one or more of the selected time intervals, with the duration.

The radiofrequency sensor may be connected to the control system, e.g. a part thereof. The control system, e.g. the part thereof, may be configured to supply a first electrical signal to the radiofrequency sensor. The radiofrequency sensor may be configured to convert the first electrical signal into the first signal. The radiofrequency sensor may be configured to convert the second signal into a second electrical signal. The second electrical signal may be received by the control system, e.g. the part thereof. The control system, e.g. the part thereof, may be configured to switch between supplying the first electrical signal to the radiofrequency sensor and receiving the second electrical signal from the radiofrequency sensor. For example, when the control system, e.g. the part thereof, has supplied the first electrical signal, the control system, e.g. the part thereof, may be configured to wait until the second electrical signal has been received, e.g. before supplying the first electrical signal again.

The system may comprise an array of radiofrequency sensors. The array of radiofrequency sensors may comprise the radiofrequency sensor. One or more radiofrequency sensors of the array of radiofrequency sensors may be configured to transmit the first signal to the part of the patient or other subject's body. One or more radiofrequency sensors of the array of radiofrequency sensors may be configured to receive the second signal from the part of the patient or other subject's body. At least one or each radiofrequency sensor of the array of radiofrequency sensors may be connected to the control system, e.g. a part thereof. The control system, e.g. the part thereof, may be configured to supply the first electrical signal to the at least one or each radiofrequency sensor of the array of radiofrequency sensors. The at least one or each radiofrequency sensor of the array of radiofrequency sensors may be configured to convert the respective first electrical signal into the first signal. The at least one, at least one other or each radiofrequency sensor of the array of radiofrequency sensors may be configured to convert the second signal into a respective second electrical signal. The second electrical signal may be received by the control system, e.g. the part thereof. The control system, e.g. the part thereof, may be configured to switch between supplying the first electrical signal to the at least one or each radiofrequency sensor of the array of radiofrequency sensors and receiving the second electrical signal from the at least one, the at least one other or each radiofrequency sensors of the array of radiofrequency sensors. The control system, e.g. the part thereof, may be configured to switch between supplying the first electrical signal to the at least one or each radiofrequency sensor of the array of radiofrequency sensors and receiving the second electrical signal from the at least one, the at least one other or each radiofrequency sensor of the array of radiofrequency sensors. For example, when the control system, e.g. the part thereof, has supplied the first electrical signal, the control system, e.g. the part thereof, may be configured to wait until the second electrical signal has been received, e.g. before supplying the first electrical signal again.

At least one or each radiofrequency sensor of the array of radiofrequency sensors may be operable between a first configuration and a second configuration. In the first configuration, the at least one or each radiofrequency sensor of the array of radiofrequency sensors may transmit the first signal or receive the second signal. The first configuration may be considered as an “on” configuration. In the second configuration, the at least one or each radiofrequency sensor of the array of radiofrequency sensors may transmit or receive no signal. The second configuration may be considered as an “off” configuration.

The system may comprise one or more switching elements. At least one or each radiofrequency sensor of the array of radiofrequency sensors may be associated with a respective switching element. The one or more or each switching element(s) may be configured to connect or disconnect the respective radiofrequency sensor of the array of radiofrequency sensors to or from the control system.

The array of radiofrequency sensors may be configured such that a phase of a first signal transmitted by at least one of the radiofrequency sensors of the array of radiofrequency sensors may be shifted relative to a phase of a first signal transmitted by at least one other of the radiofrequency sensors of the array of radiofrequency sensors. For example, the system may comprise one or more phase shifting elements. At least one or each radiofrequency sensor of the array of may be associated with and/or connected to a respective phase shifting element. The one or more or each phase shifting element(s) may be configured to change a phase of the first signal transmitted by the respective radiofrequency sensor of the array of radiofrequency sensors, e.g. relative to the phase of the first signal transmitted by the at least one other radiofrequency sensor of the array of radiofrequency sensors. This may allow the first signal transmitted by the at least one of the radiofrequency sensors of the array of radiofrequency sensors to combine or superpose with the first signal transmitted by the at least one other of the radiofrequency sensors of the array of radiofrequency sensors, e.g. to form a signal beam with increased power in a desired direction and/or to supress one or more parts of the first signals in one or more undesired directions. The signal beam, e.g. the first signals, may be steered and/or focussed, e.g. on the part of the patient or other subject's body. Alternatively or additionally, this may allow for some of the radiofrequency sensors of the array of radiofrequency sensors to be configured to transmit one or more first signals to one or more parts of the patient or other subject's body.

The radiofrequency sensor may be or comprise a second radiofrequency sensor. The system may comprise a first radiofrequency sensor. The first radiofrequency sensor may be configured to transmit a first signal to the part of the patient or other subject's body. The second radiofrequency sensor may be configured to receive a second radiofrequency signal from the part of the patient or other subject's body. The second signal may comprise a portion of the first signal that has been transmitted through the 20 part of the patient or other subject's body. The signal may be or comprise the second signal.

The system may be configured to continuously monitor the level of fluid and/or the composition of fluid in the part of the patient or other subject's body. For example, the first radiofrequency sensor may be configured to continuously transmit the first signal to the part of the patient or other subject's body. The second radiofrequency sensor may be configured to continuously receive the second signal from the part of the patient or other subject's body. The system may be configured to monitor the level of fluid and/or the composition of fluid at selected time intervals, such as every 10 minutes, every 30 minutes, every 1 hour, 2, 3, 4, 5 or 10 hours. For example, the first radiofrequency sensor may be configured to transmit the first signal to the part of the patient or other subject's body at the selected time interval. The second radiofrequency sensor may be configured to receive the second signal from the part of the patient or other subject's body at the selected time interval. The system may be configured to monitor the level of fluid and/or the composition of fluid in the part of the patient or other subject's body for a duration.

For example, the first radiofrequency sensor may be configured to transmit the first signal to the part of the patient or other subject's body during the duration. The second radiofrequency sensor may be configured to receive the second signal from the part of the patient or other subject's body during the duration. The duration may be in the range of about 5 minutes to 60 minutes, 10 minutes to 120 minutes, 1 hour to 24 hours, 12 hours to 72 hours or the like. The system may be configured to monitor the level of fluid and/or the composition of fluid at one or more intervals, e.g. one or more of the selected time intervals, with the duration.

The first and second radiofrequency sensors may be connected to the control system, e.g. the part thereof. The control system, e.g. the part thereof, may be configured to supply a first electrical signal to the first radiofrequency sensor. The first radiofrequency sensor may be configured to convert the first electrical signal into the first signal. The second radiofrequency sensor may be configured to convert the second signal into a second electrical signal. The second electrical signal may be received by the control system, e.g. the part thereof. The control system may be configured to supply the first electrical signal to the first radiofrequency sensor and receive the second electrical signal from the second radiofrequency sensor, e.g. at the same time (e.g. at substantially the same time). Alternatively, the control system, e.g. the part thereof, may be configured to switch between supplying the first electrical signal to the first radiofrequency sensor and receiving the second electrical signal from the second radiofrequency sensor. For example, when the control system, e.g. the part thereof, has supplied the first electrical signal, the control system, e.g. the part thereof, may be configured to wait until the second electrical signal has been received, e.g. before supplying the first electrical signal again.

The system may comprise a first array of radiofrequency sensors. The first array of radiofrequency sensors may comprise the first radiofrequency sensor. The system may comprise a second array of radiofrequency sensors. The second array of radiofrequency sensors may comprise the second radiofrequency sensor. One or more radiofrequency sensors of the first array of radiofrequency sensors may be configured to transmit the first signal to the part of the patient or other subject's body. One or more radiofrequency sensors of the second array of radiofrequency sensors may be configured to receive the second signal from the part of the patient or other subject's body.

At least one or each radiofrequency sensor of the first and/or second arrays of radiofrequency sensors may be connected to the control system, e.g. a part thereof. The control system, e.g. the part thereof, may be configured to supply the first electrical signal to the at least one or each radiofrequency sensor of the first array of radiofrequency sensors. The at least one or each radiofrequency sensor of the first array of radiofrequency sensors may be configured to convert the respective first electrical signal into the first signal. At least one or each radiofrequency sensor of the second array of radiofrequency sensors may be configured to convert the second signal into a respective second electrical signal. The second electrical signal may be received by the control system, e.g. the part thereof. The control system may be configured to supply the first electrical signal to the at least one or each radiofrequency sensor of the first array of radiofrequency sensors and receive the second electrical signal from the at least one or each radiofrequency sensor of the second array of radiofrequency sensors, e.g. at the same time (e.g. at substantially the same time). Alternatively, the control system, e.g. the part thereof, may be configured to switch between supplying the first electrical signal to the at least one or each radiofrequency sensor of the first array of radiofrequency sensors and receiving the second electrical signal from the at least one or each radiofrequency sensors of the second array of radiofrequency sensors.

The first and second radiofrequency sensors may be configured to be included in one or more items of furnishings or one or more other items such that the first and second radiofrequency sensors face each other and/or are on opposite sides of the part of the patient or other subject's body. The first and second radiofrequency sensors may be arranged such that the part of the patient or other subject's body is arranged between the first and second radiofrequency sensors.

The first and second arrays of radiofrequency sensors may be configured to be included in the one or more items of furnishings or the one or more other items such that the first and second arrays of radiofrequency sensors face each other and/or are on opposite sides of the part of the patient or other subject's body. The first and second arrays of radiofrequency sensors may be arranged such that the part of the patient or other subject's body is arranged between the first and second arrays of radiofrequency sensors.

At least one or each radiofrequency sensor of the first array of radiofrequency sensors may be operable between a first configuration and a second configuration. In the first configuration, the at least one or each radiofrequency sensor of the first array of radiofrequency sensors may transmit the first signal. In the second configuration, the at least one or each radiofrequency sensor of the first array of radiofrequency sensors may transmit no signal. The first configuration may be considered as an “on” configuration. The second configuration may be considered as an “off” configuration.

At least one or each radiofrequency sensor of the second array of radiofrequency sensors may be operable between a first configuration and a second configuration. In the first configuration, the at least one or each radiofrequency sensor of the second array of radiofrequency sensors may receive the second signal. In the second configuration, the at least one or each radiofrequency sensor of the second array of radiofrequency sensors may receive no signal.

At least one or each radiofrequency sensor of the first and/or second arrays of radiofrequency sensors may be associated with a respective switching element. The one or more or each switching element(s) may be configured to connect or disconnect the respective radiofrequency sensor of the first and/or second arrays of radiofrequency sensors to or from the control system.

The first array of radiofrequency sensors may be configured such that a phase of a first signal transmitted by at least one of the radiofrequency sensors of the first array of radiofrequency sensors may be shifted relative to a phase of a first signal transmitted by at least one other of the radiofrequency sensors of the first array of radiofrequency sensors. At least one or each radiofrequency sensor of the first array of radiofrequency sensors may be associated with and/or connected to a respective phase shifting element. The one or more or each phase shifting element(s) may be configured to change a phase of the first signal transmitted by the respective radiofrequency sensor of the first array of radiofrequency sensors, e.g. relative to the phase of the first signal transmitted by the at least one other radiofrequency sensor of the first array of radiofrequency sensors. This may allow the first signal transmitted by the at least one of the radiofrequency sensors of the first array of radiofrequency sensors to combine or superpose with the first signal transmitted by the at least one other of the radiofrequency sensors of first the array of radiofrequency sensors, e.g. to form a signal beam with increased power in a desired direction and/or to supress one or more parts of the first signals in one or more undesired directions. The signal beam, e.g. the first signals, may be steered and/or focussed, e.g. on the part of the patient or other subject's body. Alternatively or additionally, this may allow for some of the radiofrequency sensors of the first array of radiofrequency sensors to be configured to transmit one or more first signals to one or more parts of the patient or other subject's body.

The control system may be configured to determine the level of fluid and/or the composition of fluid in the part of the patient or other subject's body, e.g. based on the signal, e.g. the first and second signals. The control system may be configured to determine a reflection coefficient and/or a transmission coefficient, e.g. based on the signal, e.g. the first and second signals.

The control system may be configured to determine the reflection coefficient based on the first and second electrical signals. The control system may be configured to determine the transmission coefficient based on the first and second electrical signals. The first electrical signal may be proportional to or correspond to the first signal. The second electrical signal may be proportional to or correspond to the second signal.

The control system may be configured to determine the level of fluid and/or the composition of fluid in the part of the patient or other subject's body based on the determined reflection coefficient and/or transmission coefficient.

The control system may be configured to compare the determined reflection coefficient and/or transmission coefficient to one or more pre-determined reflection coefficients and/or transmission coefficients, respectively. The one or more pre-determined reflection coefficients and/or transmission coefficients may be associated with a level of fluid in the part of the patient or other subject's body and/or with a level of fluid in a part of another patient or subject's body. For example, the one or more pre-determined reflection coefficients and/or transmission coefficients may be associated with a known level of fluid in the part of the patient or other subject's body and/or with a known level of fluid in a part of another patient or subject's body.

Alternatively or additionally, the one or more pre-determined reflection coefficients and/or transmission coefficients may be associated with a composition of fluid in the part of the patient or other subject's body or with a composition of fluid in the part of the other patient or subject's body. For example, the one or more pre-determined reflection coefficients and/or transmission coefficients may be associated with a known composition of fluid in the part of the patient or other subject's body or with a known composition of fluid in the part of the other patient or subject's body. The part of the other patient or subject's body may be the same as the part of the patient or other subject's body.

The one or more pre-determined reflection and/or transmission coefficient may comprise a reflection and/or transmission coefficient that was measured for at least one of the at least two fluids, e.g. when the fluid comprises the mixture or combination of the at least two fluids. The one or more pre-determined reflection and/or transmission coefficient may comprise a reflection and/or transmission coefficient that was measured for at least one of the substance and the fluid, e.g. when the fluid comprises the mixture or combination of the fluid with the substance.

The control system may be configured to determine the level of fluid, a change of the level of fluid, the composition of fluid and/or a change of the composition of fluid in the part of the patient or other subject's body, e.g. based on the comparison between the determined reflection coefficient and/or transmission coefficient and the one or more pre-determined reflection coefficients and/or transmission coefficients, respectively. The control system may be configured to determine the level of fluid, the change of the level of fluid, the composition of fluid and/or the change of the composition of fluid in the part of the patient or other subject's body over a period of time, e.g. based on a comparison between a plurality of determined reflection coefficients and/or transmission coefficients and the one or more pre-determined reflection coefficients or transmissions coefficients, respectively.

For example, when the fluid comprises the mixture or combination of the at least two fluids, the control system may be configured to determine a concentration, e.g. a relative concentration, of the at least two fluids and/or a change of the concentration of the at least two fluids. The control system may be configured to determine the concentration, of the at least two fluids and/or the change of the concentration of the at least two fluids based on the determined reflection coefficient and/or transmission coefficient or the comparison between the determined reflection coefficient and/or transmission coefficient and the one or more pre-determined reflection coefficients and/or transmission coefficients, respectively. In this example, the terms “relative concentration” may encompass a change of an amount or level of at least one of the at least two fluids relative to at least one other of the at least two fluids.

For example, when the fluid comprises a mixture or combination of a fluid with a substance, the control system may be configured to determine a concentration, e.g. a relative concentration, of the substance and the fluid and/or a change of the concentration of the substance and the fluid. The control system may be configured to determine the concentration of the substance and the fluid and/or the change of the concentration of the substance and the fluid based on the determined reflection coefficient and/or transmission coefficient or the comparison between the determined reflection coefficient and/or transmission coefficient and the one or more pre-determined reflection coefficients and/or transmission coefficients, respectively. In this example, the terms “relative concentration” may encompass a change of an amount or level of at least one of the substance and fluid relative to at least one other of the substance and fluid.

The control system may be configured to alert a user of the system, e.g. depending on the determined level of fluid, the change of the level of fluid, the composition of fluid and/or the change of the composition of fluid in the part of the patient or other subject's body. The control system may be configured to contact an emergency service, e.g. to request an ambulance or doctor, and/or law enforcement or other official organisation, e.g. depending on the determined level of fluid, the change of the level of fluid, the composition of fluid and/or the change of the composition of fluid in the part of the patient's body.

The system may comprise an alert system. The alert system may be configured to alert the user of the system, e.g. depending on the determined level of fluid, the change of the level of fluid, the composition of fluid and/or the change of the composition of fluid in the part of the patient or other subject's body. The alert system may comprise an alarm device. The alarm device may be associated with the patient or other subject. The alarm device may comprise an optical or visual alarm device or an audio or acoustic alarm device.

The alarm device may be configured to transmit a signal to the control system. The signal may be indicative of the alert. The signal may be indicative of the determined level of fluid, the change of the level of fluid, the composition of fluid and/or the change of composition of fluid in the part of the patient or another subject's body. Additionally, the signal may be indicative of an identity of the patient or other subject and/or a location of the patient or other subject, for example in a room.

The alarm system may comprise a screen for displaying information to the user of the system. The screen may be part of the control system. Alternatively, the screen may be separate from the control system.

The information displayed on the screen may be indicative of the determined level of fluid, the change of the level of fluid, the composition of fluid and/or the change of composition of fluid in the part of the patient or other subject's body. The information displayed on the screen may be indicative of the identity of the patient or other subject and/or the location of the patient or other subject, for example in the room. The information displayed on the screen may be displayed as part of a graphical data report, such as a dashboard or the like.

The control system may use of be configured to use a machine learning model to determine or predict the level of fluid, the change of the level of fluid, the composition of fluid and/or the change of the composition of fluid in the part of the patient or other subject's body, e.g. based on the determined reflection coefficient and/or transmission coefficient. For example, the determined reflection coefficient and/or transmission coefficient may be or be considered as an input of the machine learning model. The determined or predicted level of fluid, the change of the level of fluid, the composition of fluid and/or the change of the composition of fluid in the part of the patient or other subject's body may be or be considered as an output of the machine learning model. The machine learning model may comprise or be based on a linear machine learning algorithm, such as logistic regression (LR), linear discriminant analysis (LDA) or the like, or a nonlinear machine learning algorithm, such as K-nearest neighbours (KNN), classification and regression trees (CART), Gaussian Naïve Bayes (GNB), support vector machines (SVM) or the like.

The machine learning model may be a trained machine learning model. The machine learning model may be a machine learning model that has been trained using one or more pre-determined reflection and/or transmission coefficients. The machine learning model may be a machine learning model that has been verified using one or more other pre-determined reflection and/or transmission coefficients. The pre-determined reflection and/or transmission coefficients may be or comprise one or more reflection and/or transmission coefficients determined by the control system for the patient or other subject and/or one or more other patients or other subjects, e.g. for the or a part of the body of the patient or other subject and/or the one or more other patients or other subjects. The pre-determined reflection and/or transmission coefficients may be or comprise one or more reflection and/or transmission coefficients determined by another system, apparatus or device for the patient or other subject and/or for the one or more other patients or other subjects, e.g. for the part of the body of the patient or other subject and/or the one or more other patients or other subjects.

The machine learning model may be or comprise a machine learning model that has been trained and/or verified using one or more reflection and/or transmission coefficients determined by the control system for the patient or other subject and/or the one or more other patients or other subjects, e.g. for the part of the body of the patient or other subject and/or the one or more other patients or other subjects. The machine learning model may be or comprise a machine learning model that has been trained and/or verified using one or more reflection and/or transmission coefficients determined by another system, apparatus or device for the patient or other subject and/or the one or more other patients or other subjects, e.g. for the part of the body of the patient or other subject and/or the one or more other patients or other subjects. The one or more reflection and/or transmission coefficients determined by the other system, apparatus or device may be associated with a level of fluid, e.g. a known level of fluid and/or a composition of fluid, e.g. a known composition of fluid e.g. in the part of the patient or other subject's body or the/each part of the one or more other patients or other subjects' bodies.

The radiofrequency sensor, the first radiofrequency sensor, the second radiofrequency sensor, at least one or each radiofrequency sensor of the array, first array and/or second array of radiofrequency sensors may comprise at least one of: a substrate; and a conductive element. The substrate may be or comprise a flexible or bendable substrate. The conductive element may be or comprise a flexible or bendable conductive element. By providing the radiofrequency sensor, the first radiofrequency sensor, the second radiofrequency sensor, at least one or each radiofrequency sensor of the array, first array and/or second array of radiofrequency sensors with a flexible or bendable conductive element and/or a flexible or bendable substrate, the radiofrequency sensor, the first radiofrequency sensor, the second radiofrequency sensor, at least one or each radiofrequency sensor of the array, first array and/or second array of radiofrequency sensors may be unobtrusively included in the item of furnishing or other item. In other words, this may allow for the radiofrequency sensor, the first radiofrequency sensor, the second radiofrequency sensor, at least one or each radiofrequency sensor of the array, first array and/or second array of radiofrequency sensors not to be felt by the patient or other subject using the item of furnishing or other item. Alternatively or additionally, the radiofrequency sensor, the first radiofrequency sensor, the second radiofrequency sensor, at least one or each radiofrequency sensor of the array, first array and/or second array of radiofrequency sensors may comprise at least one of: a rigid substrate; and a rigid conductive element.

The radiofrequency sensor, the first radiofrequency sensor, the second radiofrequency sensor, at least one or each radiofrequency sensor of the array, first array and/or second array of radiofrequency sensors may comprise a first cover portion and a second cover portion. The first and/or second cover portions may be flexible or bendable. The first and/or second cover portions may be configured to encase the substrate and/or the conductive element. By providing the radiofrequency sensor, the first radiofrequency sensor, the second radiofrequency sensor, at least one or each radiofrequency sensor of the array, first array and/or second array of radiofrequency sensors with first and second cover portions, the conductive element and/or substrate may be protected from contamination, fluids or like and/or washing of the item of furnishing or other item including the radiofrequency sensor, the first radiofrequency sensor, the second radiofrequency sensor, at least one or each radiofrequency sensor of the array, first array and/or second array of radiofrequency sensors may be possible.

The part of the patient or other subject's body may comprise one or more organs of the patient or other subject. The one or more organs of the patient or other subject may comprise at least one of: brain, lungs, kidneys, liver, bladder and heart.

According to a second aspect of the present disclosure there is provided an item of furnishing or other item for use by a patient or other subject comprising a fluid level and/or fluid composition monitoring system according to the first aspect. The radiofrequency sensor may be included in the item of furnishing or other item.

The item of furnishing may comprise an item of furniture for use by the patient or other subject. The item of furniture may comprise an item of furniture for the patient or other subject to sleep or rest on. The item of furniture may comprise at least one of: a bed, seat, chair and/or arm chair or the like.

The item of furnishing may comprise an item associated with furniture. The item associated with furniture may comprise at least one of: an item of bedding and a support item.

The item of furnishing may comprise a decorative item or accessory. The decorative item or accessory may comprise at least one of: a curtain and a wall hanging item.

The other item may comprise at least one of: an item or part of a vehicle, an apparatus configured to support and/or restrain a baby or infant, e.g. in a vehicle, an item or part of a shelter or dwelling and/or an item of furnishing of the shelter or dwelling.

The item or part of the vehicle may comprise an item or part of the vehicle that is in proximity to and/or surrounds the part of the patient or other subject's body to be monitored, e.g. in use. The item or part of the vehicle may comprise at least one of: a part of the vehicle configured to support the patient or other subject or a part of the patient or other subject's body, such as a seat, headrest, armrest, a fastening apparatus, such as a seat belt or one or more straps, a steering wheel, a door, a dashboard or other compartment of the vehicle, a wall and/or an item of furnishing or accessory of the vehicle.

The vehicle may comprise at least one of: an automotive vehicle, an aircraft, a spacecraft, a vehicle for transporting a baby or infant, a military vehicle and/or a space station.

The shelter of dwelling may comprise a collapsible and/or temporary shelter, such as a tent. Alternatively or additionally, the shelter of dwelling may comprise a cabin or the like. The item or part of the shelter or dwelling may comprise an item or part of the shelter or dwelling that is in proximity to and/or surrounds the part of the patient or other subject's body to be monitored, e.g. in use. The item or part of the shelter or dwelling may comprise one or more walls of the shelter or dwelling.

The item of furnishing of the shelter or dwelling may comprise an item for supporting a patient or other subject's body. The item of furnishing of the shelter or dwelling may comprise an item of furniture or a foldable item of furniture, such as a camping bed, camping chair or the like.

The radiofrequency sensor may be included in the item of furnishing or other item so as to be in proximity to the part of the patient or other subject's body to be monitored. The item of furnishing or other item may comprise the alarm device.

The item of furnishing may comprise an item of furnishing for use in an outdoor or sports environment. For example, the item of furnishing may be arranged or arrangeable in the outdoor or sports environment, e.g. in use. The outdoor or sports environment may comprise a sports or outdoor facility. The sports or outdoor facility may also be referred to as a sports or outdoor venue. The sports or outdoor facility may comprise a medical area, a medical facility, a changing area or room, and/or the like. The item of furnishing may comprise an item of furniture. The item of furniture may comprise a seat, such as a bench, chair and/or the like, bed, or other item of furniture.

According to a third aspect of the present disclosure there is provided a fluid level and/or fluid composition monitoring system comprising a radiofrequency sensor configured to be included in an outdoor or sports item or an outdoor or sports area, and a control system configured to receive a signal from the radiofrequency sensor and to determine a level of fluid and/or a composition of fluid in a part of a patient or other subject's body in vivo based on the signal. The system may comprise any of the features of the system according to the first aspect.

According to a fourth aspect of the present disclosure there is provided an outdoor or sports item or an outdoor or sports area comprising a fluid level and/or fluid composition monitoring system according to the third aspect, wherein the radiofrequency sensor is included in the outdoor or sports item or the outdoor or sports area. The outdoor or sports item or the outdoor or sports area may comprise an outdoor or sports item or an outdoor or sports area that is located or locatable in proximity to the patient or other subject, e.g. in use. For example, the outdoor or sports item or the outdoor or sports area may be located or locatable relative to the patient or other subject such that the patient or other subject is within a range of the radiofrequency sensor, e.g.

in use. The range of the radiofrequency sensor may be between about 1 meter and about 2 meters. The outdoor or sports item or the outdoor or sports area may be arranged or arrangeable in proximity to a part of the body of the patient or other subject to be monitored. The outdoor or sports item or the outdoor or sports area may be arranged or arrangeable to be separate from the patient or other subject.

The outdoor or sports item may comprise outdoor or sports equipment. The outdoor or sports item may comprise a post, goal post, a fence, a gate or the like. The outdoor or sports area may comprise a surface. The surface may comprise a playing surface, such as a pitch or court, a running surface, such as a running track, a cycling surface, such as a cycling track, and/or the like. The radiofrequency sensor may be included in the surface of the outdoor or sports area. The sports area may comprise an indoor or outdoor sports area.

The outdoor or sports item or outdoor or sports area may be part of or provided for use in an outdoor or sports environment. The outdoor or sports environment may comprise a sports or outdoor facility. The sports or outdoor facility may also be referred to as a sports or outdoor venue.

According to a fifth aspect of the present disclosure there is provided a method of determining a level of fluid and/or a composition of fluid comprising receiving data associated with or representing a reflection coefficient and/or a transmission coefficient from a part of a fluid level and/or fluid composition monitoring system according to the first or third aspect and determining the level of fluid and/or the composition of fluid in a part of a patient or other subject's body based on the data associated with or representing the reflection coefficient and/or or the transmission coefficient and/or data associated with or representing one or more pre-determined reflection coefficients and/or transmission coefficients.

The reflection coefficient and/or the transmission coefficient may be determined by the part of the control system of the fluid level and/or fluid composition monitoring system, e.g. based on the signal, e.g. the first and second signals.

The method may comprise comparing the data associated with or representing the determined reflection coefficient and/or transmission coefficient to the data representing the one or more pre-determined reflection coefficients and/or transmission coefficients, respectively.

The method may comprise determining the level of fluid and/or the composition of fluid in the part of the patient or other subject's body, e.g. based on the comparison between the data associated with or representing the reflection coefficient and/or transmission coefficient and the data associated with or representing the one or more pre-determined reflection coefficients and/or transmission coefficients, respectively.

Determining the level of fluid and/or the composition of fluid in the part of the patient or other subject's body may comprise predicting the level of fluid and/or the composition of fluid in the part of a patient or other subject's body, e.g. based on the data associated with or representing the reflection coefficient and/or or the transmission coefficient and/or the data associated with or representing the one or more pre-determined reflection coefficients and/or transmission coefficients. For example, the method may comprise using a machine learning model to determine or predict the level of fluid and/or the composition of fluid in the part of the patient or other subject's body, e.g. based on the data associated with or representing the determined reflection coefficient and/or transmission coefficient. The data associated with or representing the one or more pre-determined reflection coefficients and/or transmission coefficients may be used to train the machine learning model. The data associated with or representing the one or more pre-determined reflection coefficients and/or transmission coefficients may be used to verify the machine learning model.

According to a sixth aspect of the present disclosure there is provided a data processing device comprising a processor configured to perform the method according to the fifth aspect.

According to a seventh aspect of the present disclosure there is provided a computer program comprising instructions which, when the program is executed by a computer or data processing device, cause the computer or data processing device to carry out the method according to the fifth aspect.

According to an eight aspect of the present disclosure there is provided a computer-readable medium having stored thereon the computer program according to the seventh aspect.

According to a ninth aspect of the present disclosure there is provided an in vivo method of monitoring a level of fluid and/or a composition of fluid, the method comprising providing a fluid level and/or fluid composition monitoring system according to the first or third aspect, wherein the radiofrequency sensor is included in the item of furnishing or other item, the item of furnishing or other item being used by the patient or other subject, or wherein the radiofrequency sensor is included in the outdoor or sports item or the outdoor or sports area, receiving a signal from the radiofrequency sensor and determining a level of fluid and/or a composition of fluid in the part of the patient or other subject's body based on the signal.

The signal may comprise a portion of another signal that has been reflected by the part of the patient or other subject's body or that has been transmitted through the part of the patient or other subject's body. The other signal may be or comprise the first signal. The signal may be or comprise the second signal.

The method may comprise transmitting the first signal to the part of the patient or other subject's body. The method may comprise receiving the second signal from the part of the patient or other subject's body. The first signal may be transmitted by the radiofrequency sensor, the first radiofrequency sensor, one or more radiofrequency sensors of the array of radiofrequency sensors and/or one or more radiofrequency sensors of the first array of radiofrequency sensors. The second signal may be received by the radiofrequency sensor, the second radiofrequency sensor, one or more radiofrequency sensors of the array of radiofrequency sensors and/or one or more radiofrequency sensors of the second array of radiofrequency sensors.

The method may comprise determining a reflection coefficient and/or a transmission coefficient, e.g. based on the signal, e.g. the first and second signals.

The method may comprise any of the steps of the method according to the third aspect, e.g. to determine the level of fluid and/or the composition of fluid based on the reflection coefficient or transmission coefficient, e.g. data associated with or representing the reflection coefficient or transmission coefficient.

Various aspects and features of the present disclosure set out above or below may be combined with various other aspects and features of the present disclosure as will be readily apparent to the skilled person.

### BRIEF DESCRIPTION OF THE DRAWINGS

Some preferred embodiments of the disclosure will now be described by way of example only and with reference to the accompanying drawings, in which:

FIG. 1 depicts an exemplary embodiment of a fluid level and/or fluid composition monitoring system;

FIG. 2A depicts an exemplary item of furnishing comprising the system of FIG. 1;

FIG. 2B depicts a plan view of the item of furnishing of FIG. 2A;

FIG. 3 depicts another exemplary fluid level and/or fluid composition monitoring system;

FIG. 4 depicts an exemplary item of furnishing comprising the system of FIG. 3;

FIG. 5 depicts another exemplary a fluid level and/or fluid composition monitoring system;

FIG. 6 depicts another exemplary a fluid level and/or fluid composition monitoring system;

FIG. 7 depicts an exemplary item of furnishing comprising the system of FIG. 5 or 6;

FIG. 8 depicts another exemplary fluid level and/or fluid composition monitoring system;

FIG. 9 depicts an exemplary item of furnishing comprising the system of FIG. 8;

FIG. 10 depicts a graph of modelled SH-parameters over frequency for different levels of fluid in a part of a patient's body;

FIG. 11 depicts a graph of modelled S₂₁-parameters over frequency for different levels of fluid in a part of a patient's body;

FIGS. 12A to 12D depict measured S₁₁-parameters over frequency for different levels of fluid in a part of a patient's body;

FIG. 13 depicts measured S₂₁-parameters over frequency for different levels of fluid in a part of a patient's body;

FIG. 14 depicts the measured S₁₁-parameters of FIGS. 12A to 12D in the time-domain;

FIG. 15 depicts the measured S₂₁-parameters of FIG. 13 in the time-domain;

FIGS. 16A to 16D each depict a graph of a measured S₁₁-parameter;

FIG. 17 depicts a graph of a level of fluid measured over a period of five days;

FIGS. 18A to 18C each depict a graph of measured S₁₁-parameters over frequency for different levels of fluid in a part of a patient's body;

FIG. 19 depicts box plots of a distribution of data points of the measured S₁₁-parameters of FIGS. 18A to 18C;

FIG. 20 depicts a box plot of a distribution of a determined average accuracy value for different machine learning models;

FIGS. 21A to 21C depict an exemplary embodiment of a radiofrequency sensor for use in a fluid level and/or fluid composition monitoring system according to any one of FIG. 1, 3, 5, 6 or 8;

FIGS. 22A to 22D depict another exemplary embodiment of a radiofrequency sensor for use in a fluid level and/or fluid composition monitoring system according to any one of FIG. 1, 3, 5, 6 or 8;

FIGS. 23A to 23C depict another exemplary embodiment of a radiofrequency sensor for use in a fluid level and/or fluid composition monitoring system according to any one of FIG. 1, 3, 5, 6 or 8;

FIG. 24 depicts an exemplary vehicle comprising a fluid level and/or fluid composition monitoring system according to any one of FIG. 1, 3, 5, 6 or 8;

FIG. 25 depicts another exemplary vehicle comprising a fluid level and/or fluid composition monitoring system according to any one of FIG. 1, 3, 5, 6 or 8;

FIG. 26 depicts an exemplary shelter or dwelling and an item of furnishing of the shelter or dwelling comprising a fluid level and/or fluid composition monitoring system according to any one of FIG. 1, 3, 5, 6 or 8;

FIG. 27 depicts an exemplary item comprising a fluid level and/or fluid composition monitoring system according to any one of FIG. 1, 3, 5, 6 or 8;

FIG. 28 depicts another exemplary vehicle comprising a fluid level and/or fluid composition monitoring system according to any one of FIG. 1, 3, 5, 6 or 8;

FIG. 29 depicts another exemplary item of furnishing comprising a fluid level and/or fluid composition monitoring system according to any one of FIG. 1, 3, 5, 6 or 8;

FIG. 30 schematically depicts an exemplary alert system for use in or with the a fluid level and/or fluid composition monitoring system according to any one of FIG. 1, 3, 5, 6 or 8;

FIG. 31 depicts an exemplary an outdoor or sports item and an exemplary outdoor or sports area comprising a fluid level and/or fluid composition monitoring system according to any one of FIG. 1, 3, 5, 6 or 8;

FIG. 32 depicts an exemplary flow diagram outlining the steps of a method of determining a level of fluid and/or a composition of fluid; and

FIG. 33 depicts an exemplary flow diagram outlining the steps of an in vivo method of monitoring a level of fluid and/or a composition of fluid.

### DETAILED DESCRIPTION

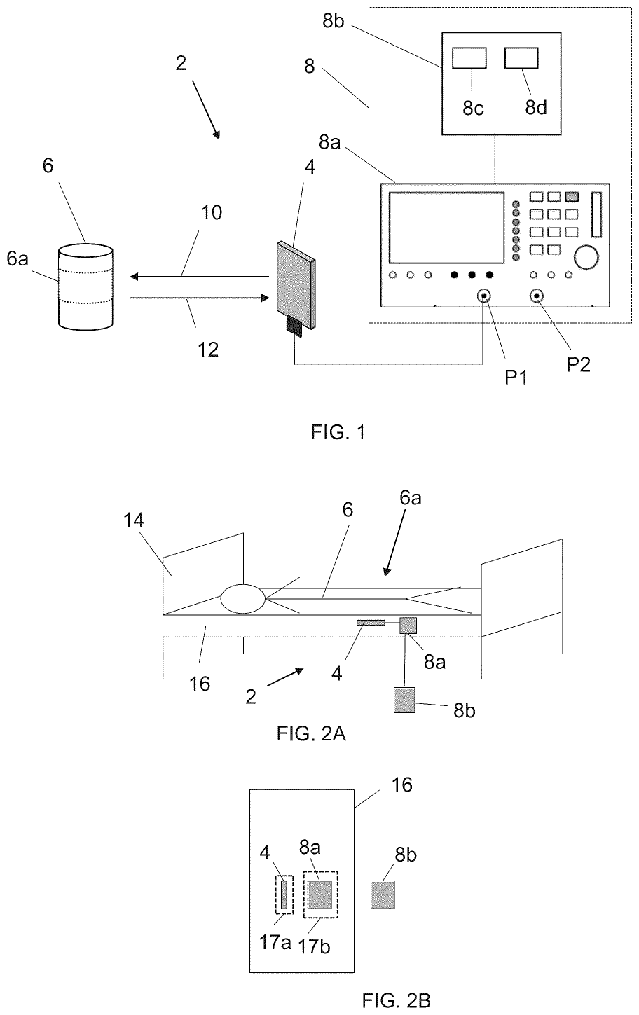

FIG. 1 shows an exemplary embodiment of a fluid level and/or fluid composition monitoring system 2. The system 2 comprises a radiofrequency sensor 4 configured to be included in an item of furnishing or another item for use by a patient or another subject. The radiofrequency sensor 4 may be configured to be embedded or integrated in the item of furnishing or other item.

By configuring the radiofrequency sensor 4 to be included in the item of furnishing or other item, a level of fluid and/or a composition of fluid in a part 6a of the patient's body 6 may be unobtrusively and/or noninvasively monitored in vivo. This may also allow the system 2 to be used in any care environment, medical environment, home or domestic environment or other environment.

The system 2 described herein may be used in a medical environment, such as a hospital, a care environment, such as a care home or other care facility, or the like. The system 2 may additionally or alternatively be used in a home or domestic environment. For example, the patient may be a patient of a care home, other care facility, hospital or the like. The other subject may be an occupant or owner of a house, flat, room or the like. In some embodiments, the patient or other subject may be a baby, infant or child. The following description refers to a patient. However, it will be appreciated that the system disclosed herein may be used for monitoring a level of fluid and/or a composition of fluid in a part of a body of another subject or another person and/or in an environment other than a medical, home or domestic environment. For example, in other embodiments, the system disclosed herein may be used for monitoring a level of fluid and/or a composition of fluid in a part of a body of a subject or a person in a business environment or work environment, such as an office or the like, or a factory environment or a manufacturing environment or the like.

In yet other embodiments, the system disclosed herein may be used for monitoring a level of fluid and/or a composition of fluid in a part of a body of a subject or a person in an outdoor environment or sports environment. This may allow for monitoring of the level of fluid and/or the composition of fluid in the part of the body of the subject or the person during a sports competition, sports game, sports race, outdoor activity and/or the like. The outdoor or sports environment may comprise a sports or outdoor facility. The sports or outdoor facility may also be referred to as a sports or outdoor venue. The sports or outdoor facility may comprise a medical area, a medical facility, a changing area or room, and/or the like.

The item of furnishing may comprise an item of furniture, such as an item of furniture for the patient to sleep or rest on. For example, the item of furniture may comprise a bed, seat, chair, armchair or the like. It will be appreciated that in some embodiments, the item of furniture may comprise an item of furniture for a baby, infant or child, such as bed, chair or other item for use by the baby, infant or child. The radiofrequency sensor 4 may be included in a part of the item of furniture. For example, the radiofrequency sensor 4 may be included in a frame of the item of furniture, a base or board of the item of furniture or the like.

The item of furnishing may comprise an item associated with furniture. For example, the item associated with furniture may comprise bedding, such as a duvet, duvet cover, sheet, pillow or the like, and/or a support item, such as a back support, mattress, cushion, headrest or the like. The item of furnishing may comprise a decorative item or accessory. For example, the decorative item or accessory may comprise a curtain, blind, a wall hanging item, such as a tapestry, picture, other decorative object or the like. The radiofrequency sensor 4 may be included in one or more parts of the item of furnishing.

The other item may comprise an item or part of a vehicle, such as an automotive vehicle, an aircraft, a spacecraft, a military vehicle, a space station, a vehicle for transporting a baby or infant or other vehicle, and/or an apparatus configured to support and/or restrain a baby, infant or child in the vehicle, such as a seat or car seat or the like.

In some embodiments, the other item comprises a shelter or dwelling, such as a collapsible and/or temporary shelter. For example, the shelter or dwelling may comprise a tent or the like. In some embodiments, the shelter or dwelling may comprise a cabin. In some embodiment, the item of furnishing or the other item comprises an item of furnishing or the shelter or dwelling.

It will be appreciated that in other embodiments, the system may be part of an outdoor or sports item or an outdoor or sports area, as will be described below.

The system 2 may comprise a control system 8. The control system 8 is configured to receive a signal from the radiofrequency sensor 4. The control system 8 is configured to determine a level of fluid and/or a composition of fluid in a part 6a of the patient's body 6 in vivo based on the signal. The signal may comprises a portion of another signal that has been reflected by the part 6a of the patient's body 6. For example, the signal may be or comprise a reflected signal. The other signal is referred to in the following as a first signal 10 and the signal is referred to in the following as a second signal 12. The first and second signals 10, 12 may each be a radiofrequency signal comprising electromagnetic waves having a frequency in the range of 20 KHz to 300 GHz, such as 300 MHz to 300 GHz, e.g. 100 MHz to 4 GHz. For example, the first and second signals 10, 12 may each have a frequency between about 1 GHz and 50 GHz or between about 1 GHz and 100 GHz.

The radiofrequency sensor 4 may be configured to transmit the first signal 10 to the part 6a of the patient's body 6. The first signal 10 may interact with the part 6a of the patient's body 6 and at least a portion of the first signal 10, which is part of the second signal 12, may be reflected by the part of the patient's body 6. The radiofrequency sensor 4 may be configured to receive the second signal 12 from the part 6a of the patient's body 6.

The system 2 may be configured to continuously monitor the level of fluid and/or the composition of fluid in the part 6a of the patient's body 6. For example, the radiofrequency sensor 4 may be configured to alternately transmit the first signal 10 to the part 6a of the patient's body 6 and receive the second signal 12 from the part 6a of the patient's body 6. The system 2 may be configured to monitor the level of fluid and/or the composition of fluid at selected time intervals, such as every 10 minutes, every 30 minutes, every 1 hour, 2, 3, 4, 5 or 10 hours. For example, the radiofrequency sensor 4 may be configured to transmit the first signal 10 to the part 6a of the patient's body 6 and to receive the second signal 12 from the part 6a of the patient's body 6 at the selected time interval.

The system 2 may be configured to monitor the level of fluid and/or the composition of fluid in the part 6a of the patient's body 6 for a duration. For example, the radiofrequency sensor 4 may be configured to alternately transmit the first signal 10 to the part 6a of the patient's body 6 and receive the second signal 12 from the part 6a of the patient's body 6 during the duration. The duration may be in the range of about 5 minutes to 60 minutes, 10 minutes to 120 minutes, 1 hour to 24 hours, 12 hours to 72 hours or the like. The system 2 may be configured to monitor the level of fluid and/or the composition of fluid at one or more intervals, e.g. one or more of the selected time intervals, with the duration.

The radiofrequency sensor 4 may be connected to the control system 8, e.g. a part thereof. For example, the radiofrequency sensor 4 may be connected to the control system 8, e.g. the part thereof by a coaxial cable. The control system 8, e.g. the part thereof, may be configured to supply a first electrical signal to the radiofrequency sensor 4, which converts the first electrical signal into the first signal 10 transmitted by the radiofrequency sensor 4. The radiofrequency sensor 4 may be configured to convert the second signal into a second electrical signal, which is received by the control system 8, e.g. the part thereof. The control system 8, e.g. the part thereof, may be configured to switch between supplying the first electrical signal to and receiving the second electrical signal from the radiofrequency sensor 4. For example, when the control system 8, e.g. the part thereof, has supplied the first electrical signal, the control system 8, e.g. the part thereof, may be configured to wait until the second electrical signal has been received, before supplying the first electrical signal again.

The control system 8 may comprise a controller 8a, which may be considered as the part of the control system 8 described above. The controller 8a may be provided in the form of a vector network analyser (VNA), such as a portable vector network analyser or a multi-port vector network analyser. The controller 8a may be configured to determine one or more S-parameters based on the first and second electrical signals, which correspond to the first and second signals 10, 12, respectively. As such, the terms “based on the first and second electrical signals” and “based on the first and second signals 10, 12” may be interchangeably used herein. The controller 8a may comprise a first port P1 and a second port P2. In the embodiment shown in FIG. 1, the radiofrequency sensor 4 is connected to the first port P1 of the controller 8a. The controller 8a may be configured to determine an S-parameter for a one-port electrical network. In this embodiment, the controller 8a may be configured to determine the S₁₁-parameter based on the first and second signals 10, 12. The S₁₁-parameter may also be considered as a reflection coefficient. As such, the terms S₁₁-parameter and reflection coefficient may be interchangeably used. The reflection coefficient or the S₁₁-parameter may correspond to a ratio between the first signal 10 and the second signal 12.

The control system 8 may comprise a data processing device 8b, which will be described in more detail below. The controller 8a may be connected to the device 8b, e.g. to transmit data representing the determined reflection coefficient to the device 8b.

FIG. 2A shows an exemplary item of furnishing comprising the system 2 shown In FIG. 1. FIG. 2B shows a plan view of the item of furnishing shown in FIG. 2A. In the embodiment shown in FIGS. 2A and 2B, the item of furnishing is provided in the form of a bed 14. The bed 14 comprises a mattress 16. In this embodiment, the radiofrequency sensor 4 is included, e.g. embedded, in the mattress 16. It will be appreciated that in other embodiments, the radiofrequency sensor may be included in another part of the bed, such as a mattress over layer or under layer, a mattress protectable cover or sheet, a frame of the bed, a base or board of the bed, a base layer of the bed, another mat or part of the mattress or the like. Alternatively, the radiofrequency sensor may be included In another item of furnishing. The other item of furnishing may comprise an item associated with the bed, such as a sheet or duvet cover.

The radiofrequency sensor 4 may be included in the item of furnishing so as to be in proximity to the part 6a of the patient's body 6 to be monitored. For example, in this embodiment, the radiofrequency sensor 4 is included in a recess 17a of the mattress 16.

The controller 8a may also be included in a respective recess 17b of the mattress 16. It will be appreciated that it may not necessary to include the radiofrequency in a recess of an item of furnishing. In other examples, the radiofrequency sensor may be arranged under a cover, on or at a frame, on a base, base layer or base board, or in a filling of the item of furnishing.

The part 6a of the patient's body 6 to be monitored may include one or more organs of the patient, such as the liver, kidneys, stomach brain, heart, bladder or lungs or the like. In this embodiment, the radiofrequency sensor 4 is included or arranged in mattress 16 so as to be in proximity to a part 6a of the patient's body 6 that includes the liver, kidneys and stomach.

The controller 8a may be connected to the device 8b, which may be arranged to be separate from the bed 14.

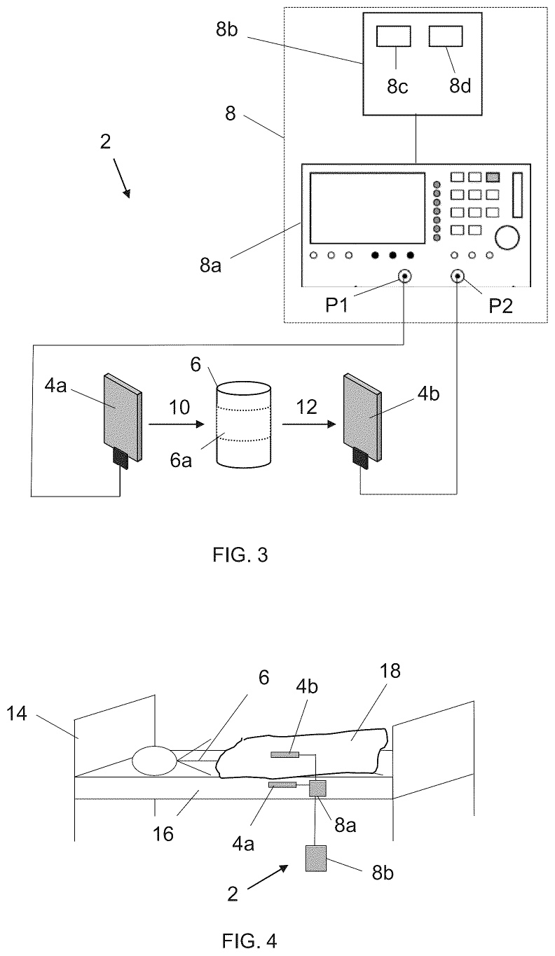

FIG. 3 shows another exemplary fluid level and/or fluid composition monitoring system 2. The system 2 shown in FIG. 3 is similar to the system 2 shown in FIG. 1. The system shown in FIG. 3 may comprise any of the features of the system shown in FIG. 1. Only differences will be described in the following. In the exemplary embodiment shown in FIG. 3, the system 2 comprises a first radiofrequency sensor 4a and a second radiofrequency sensor 4b. The first and second radiofrequency sensors 4a, 4b may be arranged on opposite sides of the part 6a of the patient's body 6.

In this embodiment, the first radiofrequency sensor 4a is configured to transmit the first signal 10 and the second radiofrequency sensor 4b is configured to receive the second signal 12. The second signal 12 may comprise a portion of the first signal 10 that has been transmitted through the part 6a of the body 6 of the patient. For example, the second signal may be or comprise a transmitted signal.

The system 2 may be configured to continuously monitor the level of fluid and/or the composition of fluid in the part 6a of the patient's body 6. For example, the first radiofrequency sensor 4a may be configured to continuously transmit the first signal 10 to the part 6a of the patient's body 6. The second radiofrequency sensor 4b may be configured to continuously receive the second signal 12 from the part 6a of the patient's body 6. The system 2 may be configured to monitor the level of fluid and/or the composition of fluid at selected time intervals, such as every 10 minutes, every 30 minutes, every 1 hour, 2, 3, 4, 5 or 10 hours. For example, the first radiofrequency sensor 4a may be configured to transmit the first signal 10 to the part 6a of the patient's body 6 at the selected time interval. The second radiofrequency sensor 4b may be configured to receive the second signal 12 from the part of the patient's body 6 at the selected time interval.

The system 2 may be configured to monitor the level of fluid and/or the composition of fluid in the part 6a of the patient's body for a duration. For example, the first radiofrequency sensor 4a may be configured to transmit the first signal 10 to the part 6a of the patient's body 6 during the duration. The second radiofrequency sensor 4b may be configured to receive the second signal 12 from the part 6a of the patient's body during the duration. The duration may be in the range of about 5 minutes to 60 minutes, 10 minutes to 120 minutes, 1 hour to 24 hours, 12 hours to 72 hours or the like. The system 2 may be configured to monitor the level of fluid and/or the composition of fluid at one or more intervals, e.g. one or more of the selected time intervals, with the duration.

The first and second radiofrequency sensors 4a, 4b may be connected to the controller 8a. The controller 8a may be configured to supply a first electrical signal to the first radiofrequency sensor 4a. The first radiofrequency sensor 4a may be configured to convert the first electrical signal into the first signal 10. The second radiofrequency sensor 4b may be configured to convert the second signal 12 into a second electrical signal. The second electrical signal may be received by the controller 8a. The controller 8a may be configured to supply the first electrical signal to the first radiofrequency sensor 4a and receive the second electrical signal from the second radiofrequency sensor 4b, e.g. at the same time (e.g. at substantially the same time). It will be appreciated that in other embodiments, the controller may be configured to switch between supplying the first electrical signal to the first radiofrequency sensor and receiving the second electrical signal from the second radiofrequency sensor. For example, when the controller has supplied the first electrical signal, the controller may be configured to wait until the second electrical signal has been received, before supplying the first electrical signal again.

In the embodiment shown in FIG. 3, the first radiofrequency sensor 4a is connected to the first port P1 of the controller 8a and the second radiofrequency sensor 4b is connected to the second port P2. The controller 8a may be configured to determine an S-parameter for a two-port electrical network. In this embodiment, the controller 8a may be configured to determine the S₂₁-parameter based on the first and second electrical signal, which correspond to first and second signals 10, 12, respectively. The S₂₁-parameter may also be considered as a transmission coefficient. As such, the terms S₂₁-parameter and transmission coefficient may be interchangeably used. The transmission coefficient, e.g. the S₂₁-parameter, may correspond to a ratio between the first signal 10 and the second signal 12. The controller 8a may be connected to the device 8b, e.g. to transmit data associated with or representing the determined transmission coefficient to the device 8b.

FIG. 4 shows an exemplary item of furnishing comprising the system 2 shown in FIG. 3. The first and second radiofrequency sensors 4a, 4b may be configured to be included in one or more items of furnishing such that the first radiofrequency sensor faces the second radiofrequency sensor. This may allow for the first signal 10 to transmit through the part 6a of the patient's body 6. For example, the first and second radiofrequency sensors 4a, 4b may be configured to be included in the one or more items of furnishing such that the first and second radiofrequency sensors 4a, 4b are on opposite sides of the part 6a of the patient's body 6. In this embodiment, the first radiofrequency sensor 4a is included in a first item of furnishing, which is provided in the form of the mattress 16 of the bed 14. The second radiofrequency sensor 4b is included in a second item of furnishing, which is provided in the form of a duvet cover 18.

It will be appreciated that in other embodiments, the first and second radiofrequency sensors may be included in the one or more items of furnishing in a different manner. For example, in other embodiments, the first radiofrequency sensor may be arranged in a first part of an item of furnishing and the second radiofrequency sensor may be arranged in a second part of the item of furnishing. The first and second parts of the item of furnishing may be moveable, configurable and/or arranged such that so that the first and second radiofrequency sensors face each other. For example, when the item of furnishing is provided in the form of a bed, the first radiofrequency sensor may be arranged in a first part of the bed and the second radiofrequency sensor may be arranged in a second part of the bed. The first and second parts of the bed may be moveable so that the first and second radiofrequency sensors face each other. Alternatively, when the item of furnishing is provided in the form of a chair or arm chair, the first radiofrequency sensor may be included in a first part of the chair or arm chair, such as a backrest of the chair or arm chair, and the second radiofrequency sensor may be included in a second part of the chair or arm chair, such as the seat of the chair or arm chair.

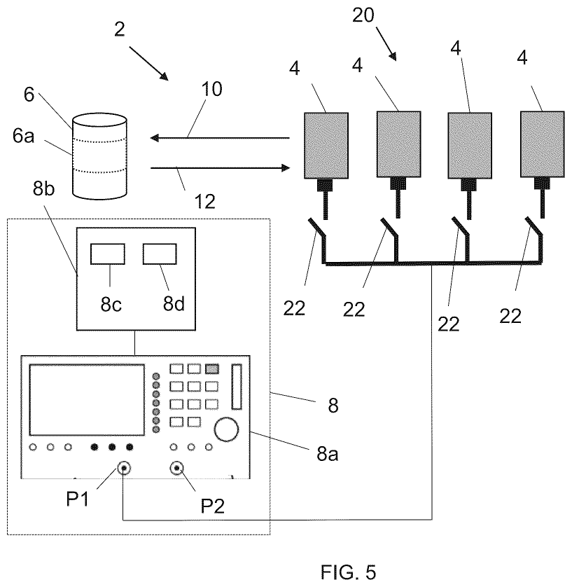

FIG. 5 shows another exemplary a fluid level and/or fluid composition monitoring system 2. The system 2 shown in FIG. 5 is similar to the system 2 shown in FIG. 1. The system 2 shown in FIG. 5 may comprise any of the features of the system 2 shown in FIG. 1. Only differences will be described in the following. In the exemplary embodiment shown in FIG. 5, the system 2 comprises an array 20 of radiofrequency sensors 4. In FIG. 5, the array 20 is shown as comprising four radiofrequency sensors 4. However, it will be appreciated that in other embodiments, the array may comprise more or less than four radiofrequency sensors.

At least one or each radiofrequency sensor 4 of the array 20 of radiofrequency sensors 4 may be configured to transmit the first signal 10 to the part 6a of the patient's body 6. The at least one, at least one other or each radiofrequency sensor 4 of the array 20 of radiofrequency sensors 4a may be configured to receive the second signal 12 from the part 6a of the patient's body 6. By providing the system 2 with an array 20 of radiofrequency sensors 4a, a larger area of the patient's body 6, e.g. the part thereof, may be monitored.

The array 20 of radiofrequency sensors 4 may be connected to the controller 8a in a same manner as the radiofrequency sensor shown in FIGS. 1 and 2. The controller 8a may be configured to supply the first electrical signal to the at least one or each radiofrequency sensors 4 of the array 20 of radiofrequency sensors. The at least one or each radiofrequency sensors 4 of the array 20 of radiofrequency sensors may be configured to convert the first electrical signal into the first signal 10. The at least one, the at least one other or each radiofrequency sensors 4 of the array 20 of radiofrequency sensors may be configured to convert the second signal 12 into the second electrical signal, which may be received by the controller 8a.

Each radiofrequency sensor 4 of the array 20 may be operable between a first configuration and a second configuration. In the first configuration, at least one or each radiofrequency sensor 4 may transmit the first signal 10 or receive the second signal 12. In the second configuration, at least one or each radiofrequency sensor 4 may transmit or receive no signal. For example, the controller 8a may be configured to operate each radiofrequency sensor 4 of the array 20 between the first and second configurations. Alternatively or additionally, the system 2 may comprise one or more switching elements 22, four of which are shown in FIG. 5. However, it will be appreciated that in other embodiments, the system may comprise more or less than four switching elements. Each radiofrequency sensor 4 of the array 20 of radiofrequency sensors may be associated with a respective switching element 22. Each switching element 22 may be configured to connect or disconnect the respective radiofrequency sensor 4 to or from the controller 8a. Each switching element 22 may be provided in the form of a switch, such as a single pole, single throw switch or the like. It will be appreciated that in other embodiments a different type of a switch than a single pole, single throw switch may be used. Each switch may be manually or wirelessly controlled, e.g. via Bluetooth. For example, by wirelessly controlling each switch, controlling of one or more radiofrequency sensors 4 of the array 20 may be facilitated. The switching elements may be part of a switching device or module. The switching device may comprise a switching time of about 5 us and a low insert loss of about up to 7 dB.

In the embodiment shown in FIG. 5, the array 20 of radiofrequency sensors 4 is connected to the first port P1 of the controller 8a. In this embodiment, the controller 8a may be configured to determine the SH-parameter based on the first and second electrical signals, which correspond to the first and second signals 10, 12, respectively.

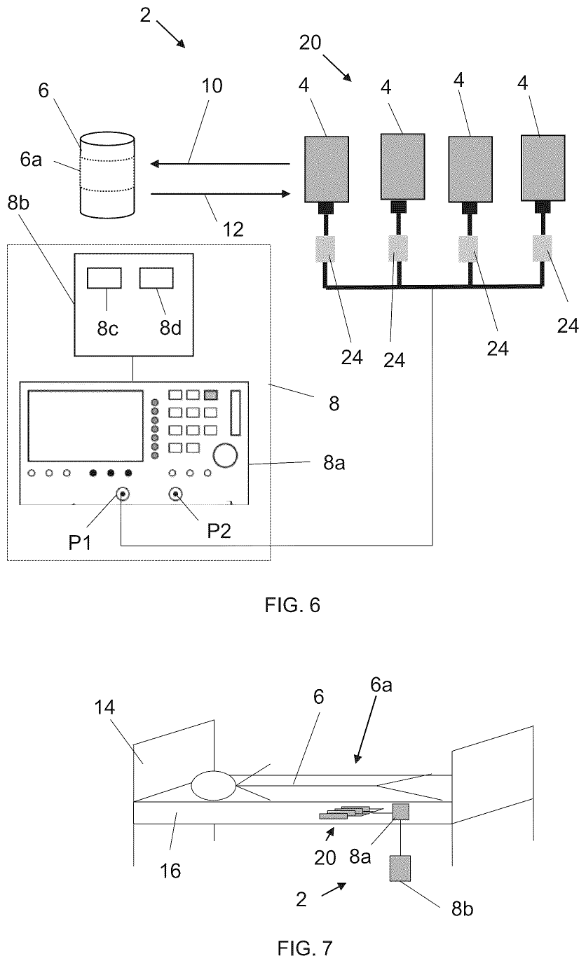

FIG. 6 shows another exemplary a fluid level and/or fluid composition monitoring system 2. The system 2 shown in FIG. 6 is similar to the system 2 shown in FIG. 5. The system 2 shown in FIG. 6 may comprise any of the features of the system 2 shown in FIG. 5. Only differences will be described in the following.

In FIG. 6, the array 20 of radiofrequency sensors 4 is configured such that a phase of the first signal transmitted by one of the radiofrequency sensors 4 of the array 20 is shifted relative to a phase of the first signal transmitted by another one of the radiofrequency sensors 4 of the array 20. For example, the array 20 of radiofrequency sensors 4 may be configured such that the phase of the first signal transmitted by the one of the radiofrequency sensors 4 of the array 20 may be shifted between 0 degrees and 360 degrees relative to the phase of the first signal transmitted by the other one of the radiofrequency sensors 4 of the array 20.

The system 2 may comprise one or more phase shifting elements 24, four of which are shown in FIG. 6. However, it will be appreciated that in other embodiments, the system may comprise more or less than four phase shifting elements. Each phase shifting element 24 may be configured to change the phase of the first signal transmitted by the one of the radiofrequency sensors 4 of the array 20 relative to the phase of the first signal transmitted by the other one of the radiofrequency sensors 4 of the array 20. The array 20 of radiofrequency sensors 4 may be considered as a phased array. By providing the system 2 with a phased array, two or more first signals 10 transmitted by two or more radiofrequency sensors 4 may combine or superpose to form a signal beam with increased power in a desired direction and/or to supress one or more parts of the signal in one or more undesired directions, e.g. while reducing a consumption of power of the array 20 of radiofrequency sensors 4. This may allow for the signal beam to be steered and/or focussed, e.g. on the part 6a of the body 6, and/or deeper organs or tissue to be monitored by the system 2. Alternatively or additionally, this may allow for some of the radiofrequency 4 sensors of the array 20 of radiofrequency sensors 4 to be configured to transmit the first signal 10 to one or more parts of the patient's body 6. One or more radiofrequency sensors 4 of the array 20 of radiofrequency sensors 4 may be configured to receive the second signal 12 from the one or more parts of the patient's body 6. It will be appreciated that a level of fluid and/or a composition of fluid in each of the one or more parts of the patient's body may be determined in the same way as described herein.

Each radiofrequency sensor 4 may be associated with and connected to a respective phase shifting element 24. Each phase shifting element 24 may be configured to change a phase of the first signal transmitted by the respective radiofrequency sensor 4. Each phase shifting element 24 may be provided in the form of a phase shifter, such as a diode phase shifter, PIN diode phase shifter, MEMS (micro-electromechanical system) phase shifter or the like. Each phase shifter may be manually or wirelessly controlled, e.g. via Bluetooth. For example, the control system 8, e.g. the processing device 8b, may be configured to control each phase shifting element 24.

FIG. 7 shows an exemplary item of furnishing comprising the system 2 shown in FIG. 5 or 6. The switching elements 22 and/or the phase shifting elements 24 have been omitted from FIG. 7 for sake of clarity. In this embodiment, the array 20 of radiofrequency sensors 4 is included in the mattress 16 of the bed 14. Reference numerals for the radiofrequency sensors have been omitted from FIG. 7 for sake of clarity.

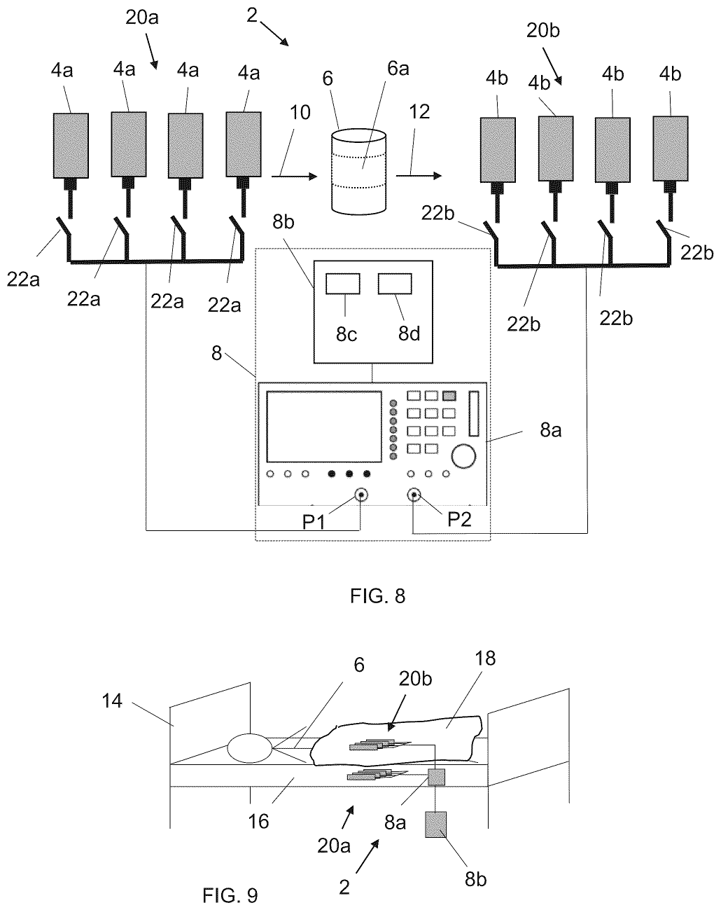

FIG. 8 shows another exemplary fluid level and/or fluid composition monitoring system 2. The system 2 shown in FIG. 6 is similar to the system 2 shown in FIG. 5. The system 2 shown in FIG. 6 may comprise any features of the system shown in FIG. 5. Only differences will be described in the following.

The system 2 may comprise a first array 20a of radiofrequency sensors 4a, four of which are shown in FIG. 8. The system 2 may comprise a second array 20b of radiofrequency sensors 4b, four of which are shown in FIG. 8. However, it will be appreciated that in other embodiments, the first and/or second arrays may each comprise more or less than four radio frequency sensors.

The first and second arrays 20a, 20b may be arranged on opposite sides of the part 6a of the body 6 of a patient. One or more radiofrequency sensors 4a of the first array 20a of radiofrequency sensors 4a may be configured to transmit the first signal 10 to the part 6a of the patient's body 6. One or more radiofrequency sensors 4b of the second array 20b of radiofrequency sensors 4b may be configured to receive the second signal 12 from the part 6a of the patient's body 6. At least one or each second signal 12 may comprise a portion of a first signal 10 that has been transmitted through the part 6a of the body 6 of the patient.

Although the first and second arrays 20a, 20b of radiofrequency sensors 4a, 4b are shown as comprising a plurality of switching elements 22a, 22b, respectively, it will be appreciated that in other embodiments, the first and/or second arrays of radiofrequency sensors may alternatively or additionally comprise a plurality of phase shifting elements, as described above in relation to FIG. 6.

The first and second arrays 20a, 20b of radiofrequency sensors 4a, 4b may be connected to the controller 8a in a same manner as the first and second radiofrequency sensors 4a, 4b shown in FIGS. 3 and 4. The controller 8a may be configured to supply the first electrical signal to at least one or each radiofrequency sensor 4a of the first array 20a of radiofrequency sensors 4a. The at least one or each radiofrequency sensor 4a of the first array 20a of radiofrequency sensors 4a may be configured to convert the first electrical signal into the first signal 10. At least one or each radiofrequency sensor 4b of the second array 20b of radiofrequency sensors 4b may be configured to convert the second signal 12 into the second electrical signal, which may be received by the controller 8a.

In the embodiment shown in FIG. 8, the first array 20a of radiofrequency sensors 4a is connected to the first port P1 of the controller 8a and the second array 20b of radiofrequency sensors 4b is connected to the second port P2. In this embodiment, the controller 8a may be configured to determine the S₂₁-parameter based on the first and second electrical signals, which correspond to the first and second signals 10, 12, respectively.

FIG. 9 shows an exemplary item of furnishing comprising the system 2 shown in FIG. 8. The first and second arrays 20a, 20b of radiofrequency sensors 4a, 4b may be configured to be included in one or more items of furnishing such that the first array 20a of radiofrequency sensors 4a faces the second array 20b of radiofrequency sensors 4b. This may allow the first signal 10 to transmit through the part 6a of the patient's body 6. For example, the first and second arrays 20a, 20b of radiofrequency sensors 4a, 4b may be configured to be included in the one or more items of furnishing such that the first and second arrays 20a, 20b of radiofrequency sensors 4a, 4b are on opposite sides of the part 6a of the patient's body 6. It will be appreciated that the first and second arrays 20a, 20b may be arranged in the one or more items of furnishing in the same manner as that described above in relation to the first and second sensors 4a, 4b.

For example, in FIG. 9, the first array 20a of the radiofrequency sensors 4a is included in the mattress 16 of the bed 14. The second array 20b of the radiofrequency sensors 4b is included in the duvet cover 18. The reference numerals for the radiofrequency sensors have been omitted from FIG. 9 for the sake of clarity. The first and second arrays 20a, 20b may be arranged such that the part 6a of the patient's body is arranged between the first and second arrays 20a, 20b.

The control system 8 may be configured to determine the level of fluid and/or the composition of fluid in part 6a of the patient's body 6 based on the first and second signals 10, 12. As described above, the control system 8, e.g. the controller 8, may be configured to determine a reflection coefficient or a transmission coefficient based on the first and second signals 10, 12.

One or more electrical properties of one or more organs and/or tissue in a human body may change with varying levels of fluid. The electrical properties of the organs and/or tissue may include the relative conductivity, electrical resistivity and/or dielectric constant, which may also be referred to as the relative permittivity. It will be appreciated that the relative conductivity may be understood as the reciprocal of the electrical resistivity. In the following description, the fluid includes water. However, it will be appreciated that in other embodiments, the fluid may comprise spinal fluid, blood and/or another body fluid. In some embodiments, the fluid may comprise a fluid other than a body fluid, such as a fluid comprising alcohol, liquid food, semisolid food and/or the like. The change in electrical properties of the organs and/or tissue may cause one or more electromagnetic waves, e.g. radiofrequency waves, to interact differently with the organs and/or tissue for different levels of fluid in the organs and/or tissue. For example, the inventors have found that the organs and/or tissue scatter electromagnetic waves, e.g.

radiofrequency waves, differently for different fluid levels therein. The determined reflection coefficient and/or transmission coefficients may be indicative of one or more changes in the electrical properties of the organs and/or tissue. As such, the determined reflection and/or transmission coefficients may be indicative of the level of fluid and/or a change of the level of fluid in the organs and/or tissue.

Alternatively or additionally, the electrical properties of the organs and/or tissue may depend on a composition of fluid that is present in the organs and/or tissue. For example, the fluid may comprise a mixture or combination of at least two fluids, e.g. at least two different fluids. Each of the two fluids may comprise different electrical properties and as such, interact differently with the electromagnetic waves, e.g.

radiofrequency waves. As a result, one or more parts or all of the determined reflection coefficient and/or transmission coefficient may be different when the fluid comprises the two different fluids compared to when the fluid comprises only one of the two fluids. This may allow for a composition of the fluid to be determined and/or the two fluids to be distinguished.

A change of an amount or level of one of the two fluids may cause a change in the determined reflection coefficient and/or transmission coefficient. This may allow for a concentration, e.g. relative concentration, of the two fluids to be determined. It will be appreciated that in some embodiments, the fluid may comprise a mixture or combination of a fluid with a substance. In such embodiments, a composition of the fluid and/or a concentration of the substance relative to the fluid may be determined in the same manner as described above in relation to the mixture or combination of the two fluids.

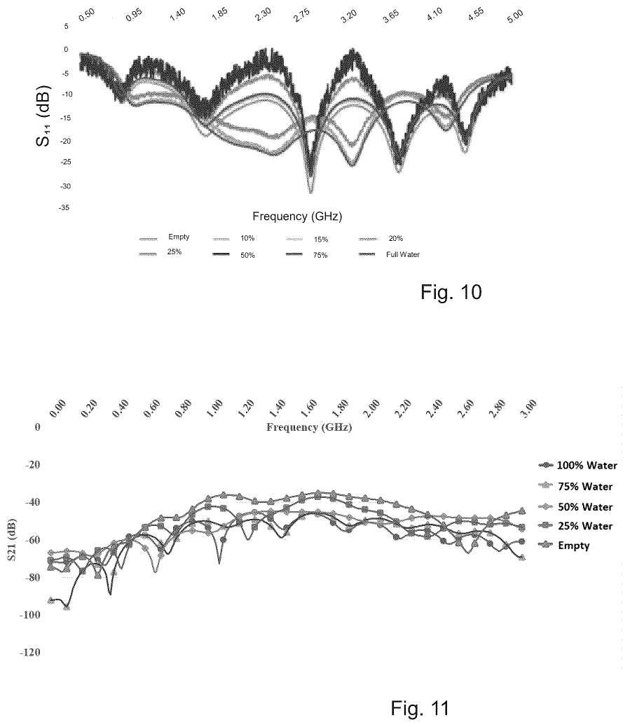

FIG. 10 shows a graph of modelled S₁₁-parameters over frequency for different levels of fluid in a part of a patient's body. The S₁₁-parameters were modelled for three radiofrequency sensors included in an item of furnishing, which in this example was provided in the form of a mattress of a bed, as shown in FIG. 7.

The Su parameters shown in FIG. 10 were modelled or simulated using a simulation and/or analysis computer program, such as CST Microwave Studio. A human model was used in the computer program and modelled to have different levels of fluid inside a plurality of organs. In this embodiment, the organs include the liver, kidneys and stomach. The different levels of fluid were modelled by changing the relative conductivity and permittivity values of the organs to correspond to the relative conductivity and permittivity values of the fluid. The Su parameters were modelled for levels of fluid ranging from 0% or empty to 100% of fluid content in the organs.

As can be seen in FIG. 10, the SH parameter, e.g. a loss of the second signal, increases with increasing level of fluid in the organs. For example, between a frequency of about 1.7 GHz and 3.7 GHz, the S₁₁ parameter changes by about −15 dB. The modelled S₁₁-parameters show that the reflection coefficient (or S₁₁-parameter) may be indicative of the level of fluid in the part 6a of the body 6 of the patient.

FIG. 11 shows a graph of modelled S₂₁-parameters over frequency for different levels of fluid in a part of a patient's body. The S₂₁-parameters shown in FIG. 11 were modelled in the same manner as those described in relation to FIG. 10. However, the S₂₁-parameters were modelled for a first radiofrequency sensor included in a first item of furnishing, which in this example was provided in the form of a mattress of a bed, and a second radiofrequency sensor included in a second item of furnishing, which in this example was provided in the form of a duvet cover or sheet, as shown in FIG. 4. As can be seen in FIG. 11, the S₂₁-parameter, e.g. the loss of the second signal, increases with increasing level of fluid in the organs. For example, between a frequency of about 0.8 GHz and 2.2 GHz, the S₂₁-parameter increases by about 60% from −40 dB to about-60 dB between 0% of fluid or empty and 100% of fluid. The modelled S₂₁-parameters show that the transmission coefficient (or S₂₁-parameter) may be indicative of the level of fluid in the part of the patient's body.

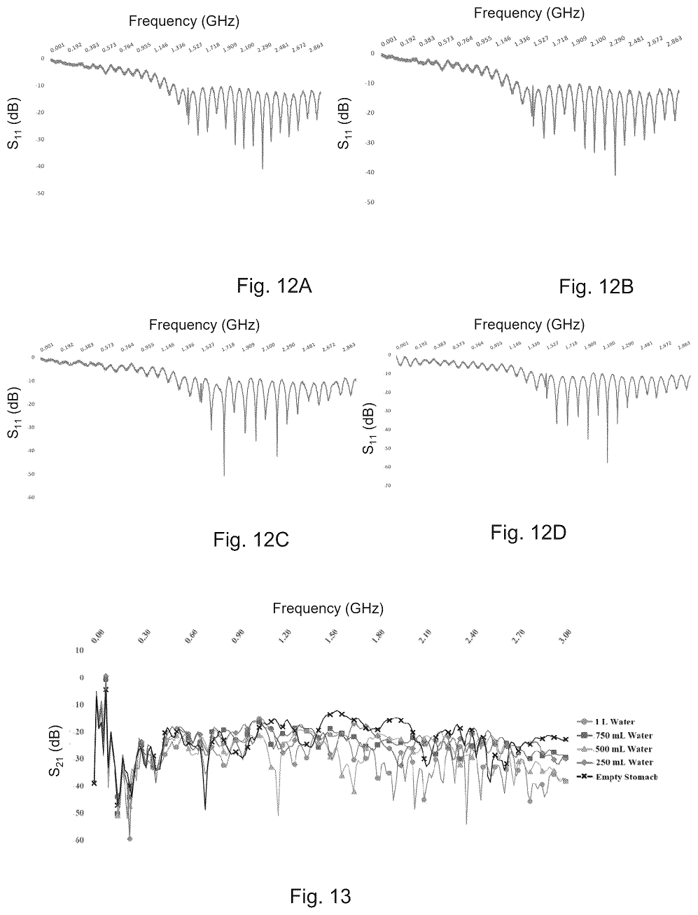

FIGS. 12A to 12D show measured S₁₁-parameters over frequency for different levels of fluid in a part of a patient's body. The SH-parameter shown in FIG. 12A was measured after a fast of 14 hours to ensure that the fluid level in the organs was a low as possible. The S₁₁-parameter shown in FIG. 12B was measured after the patient drank 300 ml of water. The SH-parameter in FIG. 12C was measured after the patient drank another 300 ml of water so that the total amount of water drank by the patient was 600 ml. The S₁₁-parameter in FIG. 12D was measured after the patient drank 400 ml of water so that the total amount of water drank by the patient was 1 litre. In this example, the item of furnishing was provided in the form of a mattress. The radiofrequency sensor was included in the mattress, as shown in FIG. 2. The radiofrequency sensor was included in the mattress to be in proximity to a part of the patient's body that includes the liver, kidneys and stomach. The SH-parameters shown in each of FIGS. 12a to 12D were averaged over ten measurements in a frequency range of 200 MHz to 3 GHz.

From FIGS. 12A to 12D, it can be seen that there is an increase in the S₁₁-parameter, e.g. in loss of the second signal, with increasing level of water in the part of the patient's body. For example, in FIG. 12A, the S₁₁-parameter is about −26 dB at about 2 GHz. In FIG. 12B, the St-parameter is about −32 dB at about 2 GHz. In FIG. 12C, the S₁₁-parameter is about −35 dB at about 2 GHz. In FIG. 12D, the S₁₁-parameter is about −45 dB at about 2 GHz. As such, it can be seen from FIGS. 12A to 12D that with increasing level of fluid in the organs, the reflection coefficient or S₁₁-parameter, e.g. the loss of the second signal, increases.

FIG. 13 shows measured S₂₁-parameters over frequency for different levels of fluid in a part of a patient's body. The S₂₁-parameters shown in FIG. 13 were measured in a similar manner to those shown in FIGS. 12a to 12D. However, a first radiofrequency sensor was included in first item of furnishing and a second radiofrequency sensor was included in second item of furnishing. In this example, the first item of furnishing was provided in the form of a mattress of a bed and the second item of furnishing was provided in the form of a duvet cover or sheet. The S₂₁-parameters shown in FIG. 13 were measured after a fast of 14 hours to ensure that the fluid level in the organs was a low as possible. The patient drank water in 250 ml increments. The S₂₁-parameters were measured for total amounts of 250 ml, 500 ml, 750 ml and 1 litre of water drank by the patient.

From FIG. 13, it can be seen that there is an increase in the S₂₁-parameter, in the loss of the second signal, with increasing level of water in the part of the patient's body. For example, between about 1.7 GHZ and 2.7 GHz, the S₂₁-parameter increases from about −20 dB to about −40 dB. As such, it can be seen from FIG. 13 that with increasing level of fluid in the organs, the transmission coefficient or S₂₁-parameter, e.g. the loss of the second signal, increases.

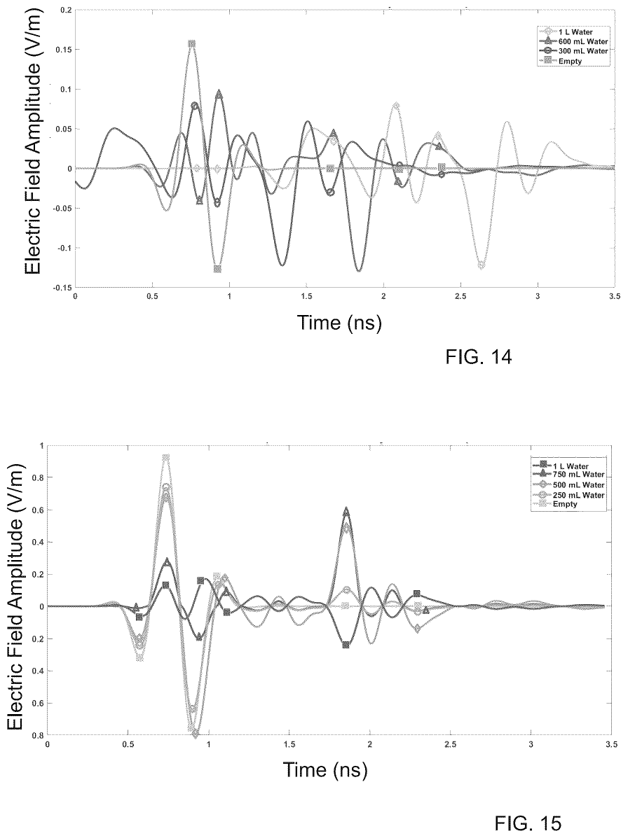

FIG. 14 shows the measured SH-parameters shown in FIGS. 12A to 12D in the time-domain. FIG. 15 shows the measured S₂₁-parameters shown in FIG. 13 in the time-domain. In FIGS. 14 and 15, the measured S₁₁-parameters and S₂₁-parameters, respectively, are expressed in terms of an electric field amplitude over time. The measured S₁₁-parameters and S₂₁-parameters may be converted from the frequency domain, as shown in FIGS. 12A to 12D and 13, to the time-domain, as shown in FIGS. 14 and 15, using a Fourier transform function, such as an Inverse Fast Fourier Transform. The conversion of the measured S₁₁-parameters and S₂₁-parameters from the frequency-domain to the time-domain may be performed by executing a computer program, such as MATLAB or the like. By converting the measured S₁₁-parameters and

S₂₁-parameters into the time-domain, it may be possible for a user of the system 2 to monitor the level of fluid in the part 6a of the body 6 of the patient in real-time. Additionally or alternatively, this may allow the system 2 to supply real-time information relating to the level of fluid and/or a change thereof in the part 6a of the patient's body 6 to device 8b or another device, such as a health monitoring device, vital sign monitoring device, a smart digital device or system, IoT (internet of things) device or system or the like.

From FIG. 14, it can be seen that the electric field amplitude changes with Increasing levels of fluid. For example, the amplitude of the peaks and troughs of the measured S₁₁-parameter decreases with increasing level of fluid. In addition, there is a shift of the peaks and troughs associated with the measured S₁₁-parameter towards increasing times for increasing levels of fluid. The shift of the peaks and troughs may be due to the increase in the measured S1-parameter. For example, an increase in the measured S₁₁-parameter in the frequency-domain may correlate to a delay of the associated signal in the time-domain. The delay may be due to the controller 8a switching the operation of the radiofrequency sensor 4 between transmitting the first signal 10 and receiving the second signal 12, as described above.

From FIG. 15, it can be seen that the electric field amplitude changes with increasing levels of fluid. For example, the amplitude of the peaks and troughs of the measured S₂₁-parameter decreases with increasing level of fluid. The decrease in the amplitude of the peaks and troughs of the measured S₂₁-parameter is larger compared to the decrease in the amplitude of the peaks and troughs of the measured S₁₁-parameter shown in FIG. 14. This may be due to an increase absorption of the first radiofrequency signal due to an increase of the level of fluid in the organs, when the first signal passes through the part 6a of the patient's body 6.

As described above, the control system 8 may comprise the controller 8a. The control system 8 may comprise the device 8b. The device 8b may comprise a processor 8c for executing instructions and one or more memory devices 8d for storing instructions and data. The instructions may be part of a computer program.

The device 8b may be connected to the controller 8a. The controller 8a may be configured to determine the reflection coefficient and/or transmission coefficient based on the first and second electrical signals, which correspond to the first and second signals 10, 12, respectively. The controller 8a may be configured to transmit the determined reflection coefficient and/or transmission coefficient to the device 8b, e.g. when requested by the device 8b.

The control system 8, e.g. the device 8b, may be configured to determine the level of fluid and/or the composition of fluid in the part 6a of the patient's body 6 based on the determined reflection coefficient and/or transmission coefficient. For example, the control system 8, e.g. the device 8b, may be configured to determine the level of fluid and/or the composition of fluid in the part 6a of the patient's body 6 based on the determined reflection coefficient and/or transmission coefficient and one or more pre-determined reflection coefficients and/or transmission coefficients.

The control system 8, e.g. the device 8b, may configured to compare the determined reflection coefficient and/or transmission coefficient to the one or more pre-determined reflection coefficients and/or transmission coefficients, respectively. The pre-determined reflection coefficients and/or transmission coefficients may be stored in the memory device 8d. The pre-determined reflection and/or transmission coefficient may be associated with a level of fluid and/or the composition of fluid in the part 6a of the patient's body 6. For example, the pre-determined reflection and/or transmission coefficient may comprise a reflection and/or transmission coefficient that was measured when a level of the fluid in the part 6a of the patient's body 6 is minimal, e.g. after a fast. Additionally or alternatively, the or each pre-determined reflection coefficient and/or transmission coefficient may associated with a level of fluid in the part 6a of the patient's body 6 that is different from the minimal level of fluid. It will be appreciated that the or each pre-determined reflection coefficient and/or transmission coefficient may be associated with a level of fluid in a part, e.g. the same part as the part 6a of the patient's body 6, of another patient or subject's body. For example. The other patient or subject may share one or more attributes with the patient, such as a height, weight and/or age or the like.

Alternatively or additionally, the pre-determined reflection and/or transmission coefficient may comprise a reflection and/or transmission coefficient that was measured for a known composition of fluid in the part 6a of the patient's body 6 or in the same part of the other patient's body. It will be appreciated that in some embodiments, the pre-determined reflection and/or transmission coefficient may comprise a reflection and/or transmission coefficient that was measured for at least one of the two fluids, e.g. when the fluid comprises a mixture or combination of the two fluids, and/or at least one of the substance and fluid, e.g. when the fluid comprises a mixture or combination of a fluid with a substance. The pre-determined reflection coefficient and/or transmission coefficient may also be referred to as calibration coefficients and/or reference coefficients.

The control system 8, e.g. the device 8b, may configured to determine the level of fluid and/or the composition of fluid in the part 6a of the body 6 of the patient based on the comparison between the determined reflection coefficient and/or transmission coefficient and the one or more pre-determined reflection coefficients and/or transmission coefficients, respectively. For example, the control system 8, e.g. the device 8b, may be configured to correlate one or more changes between the determined reflection coefficient and/or transmission coefficient and the one or more pre-determined reflection coefficients and/or transmission coefficients, respectively, to a level of fluid and/or a composition of fluid in the part 6a of the body 6 of the patient, a change of the level of fluid and/or a change of the composition of fluid in the part 6a of the patient's body.

The control system 8, e.g. the device 8b, may also be configured to determine the level of fluid, the change of the level of fluid, the composition of fluid and/or the change of composition of fluid in the part 6a of the body 6 of the patient over a period of time, e.g. based on a comparison between a plurality determined reflection coefficients and/or transmission coefficients and the one or more pre-determined reflection coefficients and/or transmissions coefficients, respectively.

For example, depending on the determined level of fluid, the change of the level of fluid, the composition of fluid and/or the change of composition of fluid in the part of the patient's body, the control system 8, e.g. the device 8b, may be configured to alert a user of the system 2, such as a doctor, carer, law enforcement or other health care professional. In some embodiments, the user of the system may be a parent of a baby, infant or child. The alert may be or comprise an alarm, an alert message, an automated alert or the like. Depending on the determined level of fluid, the change of the level of fluid, the composition of fluid and/or the change of composition of fluid in the part of the patient's body, the control system 8, e.g. the device 8b, may be configured to contact an emergency service, e.g. to request an ambulance or doctor. For example, an increase or decrease in the level of fluid in the part of the patient's body, e.g. above or below a threshold, may be indicative of a state of health, a level of hydration, a disease, a medical condition and/or a need of the patient, such as the patient's need to use a bathroom. Additionally or alternatively, the composition of fluid and/or a change thereof may be indicative of a state of health, a disease, a medical condition and/or a need of the patient. As such, the system 2 described herein may allow the for prediction of possible oncoming adverse events, such as a fall, incontinence, dehydration, a disease or other medical condition. The determined level of fluid, change of the level of fluid, the composition of fluid and/or the change of the composition of fluid may be used by a doctor or other health professional to make a diagnosis of the disease or medical condition and/or to assist the patient with their need.

The control system 8, e.g. the device 8b, may be configured to use a machine learning model to determine or predict the level of fluid, the change of the level of fluid, the composition of fluid and/or the change of composition of fluid in the part 6a of the patient's body 6 e.g. based on a determined reflection coefficient and/or transmission coefficient. The processing device 8b, may be configured to use the machine learning model to correlate the determined level of fluid, the change of the level of fluid, the composition of fluid and the change of composition of fluid in the part 6a of the patient's body 6 with the disease, medical condition and/or the need of the patient.

The machine learning model may be a trained machine learning model. The machine learning model may be based on a linear machine learning algorithm, such as logistic regression (LR), linear discriminant analysis (LDA) or the like, or a nonlinear machine learning algorithm, such as K-nearest neighbours (KNN), classification and regression trees (CART), Gaussian Naïve Bayes (GNB), support vector machines (SVM) or the like.

In order to determine which of the above described machine leaning algorithms has the highest accuracy, a machine learning model may be developed for each machine leaning algorithm. Data associated with pre-determined reflection and/or transmission coefficients may be split, e.g. randomly split, into two sets. A first set of the data may be used to train each machine learning model and a second set of the data may be used to validate at least one of the machine learning models. For example, about 80% of the data associated with pre-determined reflection and/or transmission coefficients may be used in the first set of data and the remaining 20% of the data associated with the pre-determined reflection and/or transmission coefficients may be used in the second set of data. It will be appreciated that in other embodiments a different portion than 80% and 20% of the data associated with the pre-determined reflection and/or transmission coefficients may be used in the first set of data and second set of data, respectively.

An accuracy of each machine learning model may be estimated using a validation or cross-validation method, such as the k-fold cross-validation method, on the first set of data. Based on the estimated accuracy of each machine learning model one of the machine learning models may be selected. The selected machine learning model may be used on the second set of data, e.g. to determine an accuracy of the selected machine learning model, e.g. on unseen data. In other words, the second set of data may be used to verify the selected machine learning model.

As described above, the pre-determined reflection and/or transmission coefficients may be comprised in the first and second data sets. Each of the pre-determined reflection and/or transmission coefficients may be associated with a respective level of fluid and/or a composition of fluid in the part of the patient's body or the same part of another patient or other subject's body. For example, one or more of the pre-determined reflection coefficients and/or transmission coefficients may be obtained by the patient, one or more other patients or other subjects consuming a known amount of one or more fluids at regular intervals, e.g. after a period of fasting, and determining the reflection coefficient and/or transmission coefficient after consumption of the known amount of the one or more fluids, e.g. as described above. For example, the S₁₁-parameters shown in FIGS. 12A to 12D and/or the S₂₁-parameters shown in FIG. 13 may be considered as the pre-determined reflection coefficients and/or transmission coefficients. The S₁₁-parameters shown in FIGS. 12A to 12D and/or the S₂₁-parameters shown in FIG. 13 may be comprised in the first and second data sets. It will be appreciated that the pre-determined reflection coefficient and/or transmission coefficient may have been obtained, as described above, for a plurality of patients or other subjects with different attributes, such as different heights, weights and/or ages or the like. Additionally or alternatively, the pre-determined reflection coefficients and/or transmission coefficients may have been obtained by one or more systems and/or methods different to those described herein.

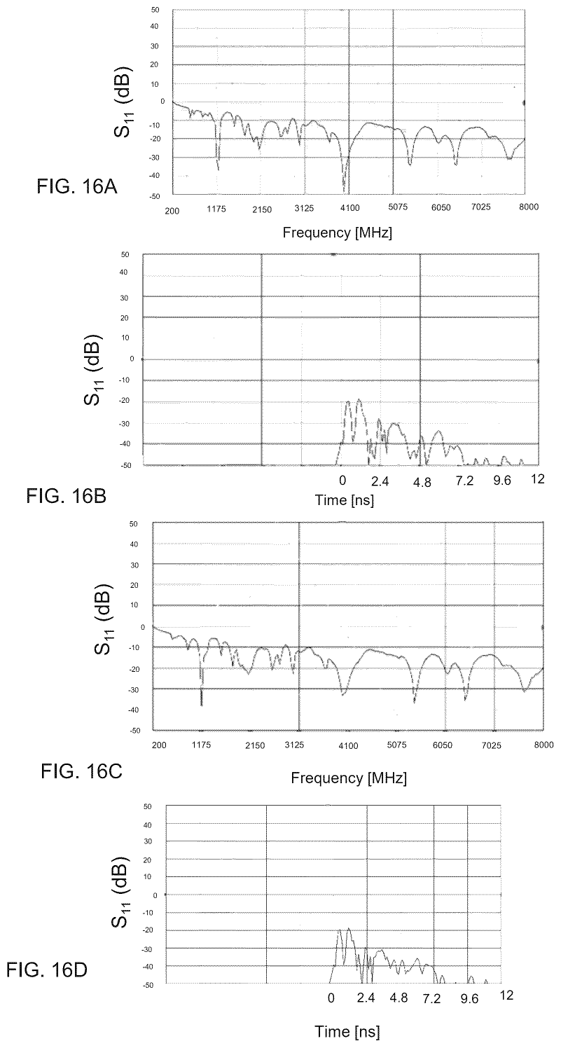

FIGS. 16A to 16D each show a graph of a measured S₁₁-parameter. FIGS. 16A and 16C each show a graph of the measured SH-parameter over frequency. FIGS. 16B and 16D each show a graph of the measured S₁₁-parameter over time. For example, FIG. 16B shows the measured SH-parameter shown in FIG. 16A in the time domain and FIG. 16D shows the measured S₁₁-parameter shown in FIG. 16C in the time domain.

For the measurements of the SH-parameter shown in FIGS. 16A to 16D, four radiofrequency sensors 4 were included in an item of furnishing, which was in the form of a cushion. The cushion was arranged relative to the patient such that the S₁₁-parameters were measured for a part of the patient's body that includes the liver, kidneys and stomach.

For the measurements shown in FIGS. 16A and 16B, the patient was resting on the cushion during the measurements and had an empty stomach. The measurements of the S₁₁-parameter can be considered as a calibration measurement of the S₁₁-parameter. The measured S₁₁-parameters shown in FIGS. 16A and 16B may be considered as at least one of the calibration coefficients and/or reference coefficients mentioned above.

FIGS. 16C and 16D show the measured S₁₁-parameter after the consumption of 1 litre of water. A described above, the patient was resting on the cushion. It can be seen from FIG. 16C that the S₁₁-parameter has changed after the consumption of the water in the range of about 4 GHz to 8 GHz. The changes of the SH-parameter are indicative of a change of the level of fluid of in the part of the patient's body. The change of the SH-parameter after the consumption of the water may be due to a change of the level of fluid in the part of the patient's body during consumption of the water. The measured S₁₁-parameters shown in FIGS. 16C and/or 16D may be used in combination with measured SH-parameters shown in FIGS. 16A and/or 16B to determine the level of fluid in the part of the patient's body.

It can be seen that the SH-parameter at about 1.2 GHz is increased in FIG. 16C relative to FIG. 16A. This may be due to movement of patient during the measurement. In this example, the patient moved his arm during the measurement of the SH-parameter. Different frequencies or frequency ranges may be associated with different parts of the body of the patient. For example, the frequency range of about 200 MHz to about 3.1 GHz may be associated with an upper part of the body of the patient, e.g. including the patient's chest, arms etc. The frequency range of about 3.1 GHz to 8 GHz may be associated with a lower part of the body of the patient, e.g. including the patient's stomach, liver, kidneys, abdomen etc.

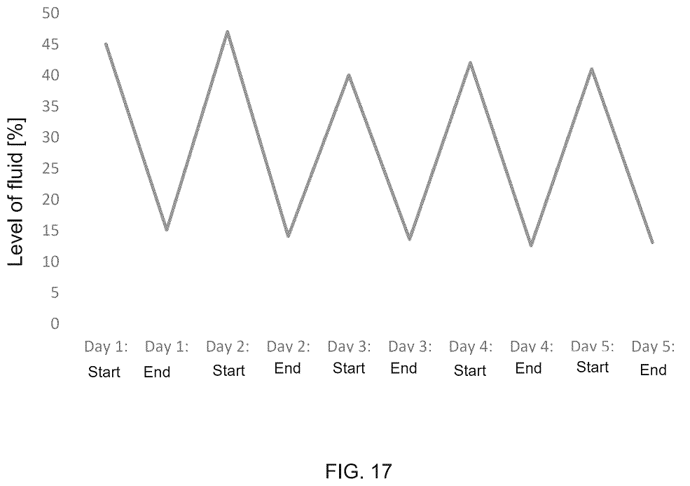

FIG. 17 shows a graph of a level of fluid measured over a period of five days. The level of fluid shown in FIG. 17 was measured during the Islamic month of fastening. The level of fluid was determined by measuring the SH-parameter, as described above. The radiofrequency sensors were included in the cushion and the cushion was arranged relative to the patient, as described in relation to FIG. 16A to 16D. Prior to the measurements shown in FIG. 17, the patient fasted 16 to 17 hours a day for a period of 20 days. Each day the level of fluid was measured at the start of the fast at around 3:30 am and the end of the fast at 8:30 pm. Prior to the measurements taken on the start of each day, the patient drank 700 mL to 1 litre of water. It can be seen from FIG. 17 that the measured level of fluid is increased at the start of the day relative to the measured level of fluid at the end of the day. The peaks in FIG. 17 are indicative of the increased level of fluid at the start of each day. The troughs in FIG. 17 are indicative of a lower level of water at the end of the day relative to the start of the day.

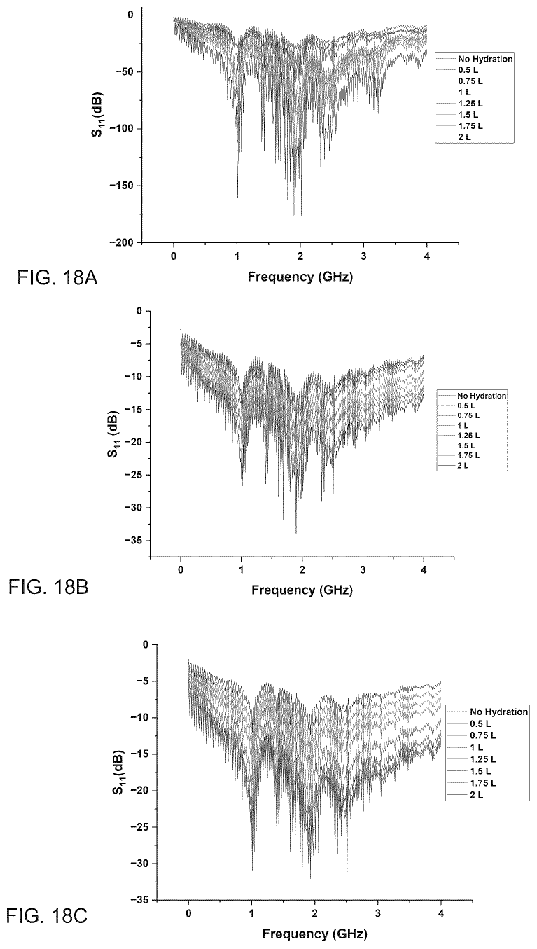

FIGS. 18A to 18C each show a graph of measured S₁₁-parameters over frequency for different levels of fluid in a part of a patient's body. The SH-parameters shown in FIGS. 18A to 18C were measured for three different patients. The S₁₁-parameters shown in FIG. 18A were measured for a first patient. The St-parameters shown in FIG. 18B were measured for a second patient. The S₁₁-parameters shown in FIG. 18C were measured for a third patient. The data shown in FIGS. 18A to 18C was measured by nine radiofrequency sensors, which were embedded into a mattress. The radiofrequency sensors were arranged in the mattress such that the S₁₁-parameters were measured for a part of the patient's body that includes the liver, kidneys and stomach. Prior to measuring the S₁₁-parameters, each patient fasted for a 14 hour period. The S₁₁-parameters were then measured for an empty stomach, which is referred to as “no hydration” in FIGS. 18A to 18C and total amounts of 500 ml, 750 ml and 1 litre, 1.25 litres, 1.5 litres and 2 litres of water, which were drank by each patient. Ten measurements were taken for each of the nine radiofrequency sensors in a frequency range of about 100 MHz to 4 GHz. FIGS. 18A to 18C show the average S₁₁-parameters for each level of consumed water.

It can be seen from FIGS. 18A to 18C that there is an increase in the S₁₁-parameter, e.g. in loss of the second signal, with increasing level of water in the part of the patient's body. For example, in FIG. 18A, the SH-parameter is about −24 dB at 2 GHz for an empty stomach and −45 dB after consumption of 1 litre of water. This may be considered as an increase of about 83% of the S₁₁-parameter, e.g. of the second signal, measured for the first patient. Similar changes in the SH-parameter were measured for the second and third patients, as can be seen in FIGS. 18B and 18C. However, it can be seen in FIGS. 18A to 18C that the change in the respective S₁₁-parameters is different for each patient. This may be due to the three patients having different body compositions. For example, the first patient was female while each the second and third patients was male. For example, at about 2 GHz, the measured S₁₁-parameter after a consumption of 2 litres of water is close to −175 dB for the first patient. This is an increase of approximately 630% of the SH-parameter, e.g. loss in the second signal, relative to the S₁₁-paramyer measured for the empty stomach. The corresponding increase of the S₁₁-parameters for each of the second and third patients was determined of about 132% and 267%, respectively.

Table 1 below summarises a statistical analysis of the data measured by the nine radiofrequency sensors, which are indicated in the table by numbers 1 to 9, for the three patients. The table includes a number of measured data points, a mean value, a minimum value and a maximum of the SH-parameter measured by each of the nine antennas for all amounts of water consumed by the three patients. The table also includes a standard deviation of each mean value.

| | 1 | 2 | 3 | 4 | 5 | 6 | 7 | 8 | 9 |
|---|---|---|---|---|---|---|---|---|---|
| Number of | 32080 | 32080 | 32080 | 32080 | 32080 | 32080 | 32080 | 32080 | 32080 |
| measured | | | | | | | | | |
| data points | | | | | | | | | |
| Mean (dB) | −17.5 | −17 | −17.6 | −18.2 | −18.1 | −18.2 | −18.7 | −19.8 | −23.4 |
| Std. Dev. | 5.87 | 6.457 | 6.275 | 9.193 | 7.658 | 8.128 | 9.435 | 12.76 | 33.17 |
| (dB) | | | | | | | | | |
| Min (dB) | −49.3 | −61.04 | −176.5 | −94.24 | −122.7 | −177.4 | −91.37 | −91.83 | −99.32 |
| Max (dB) | −2.39 | −1.33 | −1.73 | −7.57 | −2.53 | −2.79 | −2.39 | −1.99 | −2.64 |

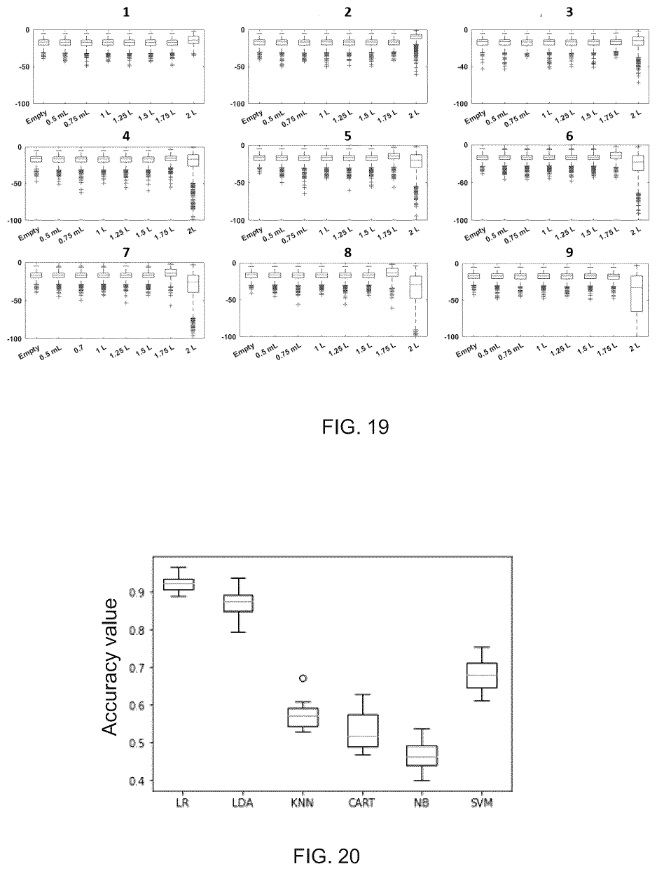

FIG. 19 depicts box plots of a distribution of data points of the measured S₁₁-parameters shown in FIGS. 18A to 180. The radiofrequency sensors are indicated by numbers 1 to 9 in FIG. 19. It can be seen from FIG. 19 that there appears to be no linear or normal distribution of the data points for each of the nine radiofrequency sensors.

The measured data shown in FIGS. 18A to 18C was used to evaluate machine learning models based on the following machine learning algorithms: Gaussian Naive Bayes (GNB), Logistic Regression (LR), Linear Discriminant Analysis (LDA) and K-Nearest Neighbors (KNN), Classification and Regression Trees (CART) and Support Vector Machines (SVM). The measured data was split into a first data set and a second data set. About 80% of the measured data was included in the first data set and about 20% of the measured data were included in the second data set. The measured data was randomly split into the first and second data sets, e.g. using a random seed number to initialise a random number generator to select rows of the data at random. The first data set may be used to train the machine learning model and the second data set may be used as a validation data set. The machine learning models were separately trained and evaluated. The random seed number was reset before training of each machine learning model so that a random seed number used for a previous machine learning model was not used again.

As described above, an accuracy of each machine learning model may be estimated using a validation or cross-validation method, such as the k-fold cross-validation method, on the first set of data. This may include randomly shuffling and splitting the first data set into k groups. One of the groups is used a test data set and the remaining groups are used as training data sets. A value of k may be 5 or 10. In this example, k was selected as 10. As such, the first data set was split into nine training data sets and one test data set. Each of the machine learning models is trained using the training data sets and evaluated on the test data set. This was repeated for all combinations of training data sets. An accuracy value may be generated to summarise an accuracy of each of the machine learning models. The accuracy value is a ratio of a number of correctly predicted instances divided by a total number of instances in the training data set dataset. This accuracy value was used to evaluate each of the machine learning models. Each of the machine learning models were trained and evaluated for a number of iterations. In this example, the number is iterations is equal to ten. The accuracy value was determined after each iteration and averaged.

FIG. 20 shows a box plot of a distribution of the determined average accuracy value for each of the machine learning models. From FIG. 20, it can be seen that for both the machine learning model based on the Logistic Regression algorithm and the machine learning model based on the Linear Discriminant Analysis algorithm an average accuracy value above 80% was determined. For the machine learning model based on the Logistic Regression algorithm, the average accuracy values was determined as 93.58%, while the average accuracy value for the machine learning model based on the Linear Discriminant Analysis algorithm was determined as 88.26%. A relationship between the data obtained for each of the nine radiofrequency sensors may be uncorrelated. As such, the machine learning model based on the Logistic Regression algorithm and the machine learning model based on the Linear Discriminant Analysis algorithm have a better performance compared to the machine learning models based on the remaining machine learning algorithms.

The accuracy of the machine learning model based on the Logistic Regression algorithm was further tested using the second data set mentioned above. This may allow an influence of factors, such as overfitting of data or a data leak, which may affect the prediction of each of these models to be reduced. Using the second data set, an accuracy of the machine learning model based on the Logistic Regression algorithm was determined as 100%. In other words, the machine learning model based on the Logistic Regression algorithm could accurately predict the level of water consumed by the patients. The Logistic Regression algorithm can be understood as analysing a relationship between one or more independent variables and classifying data into discrete classes. For example, the Logistic Regression algorithm is configured to estimate a mathematical probability of whether an instance belongs to a specific class or not. In this example, each level of fluid may be considered as a respective class. The Logistic Regression algorithm may be based on a logistic function, such as a sigmoid function.

Table 2 shows a confusion matrix that was determined for the machine learning model based on the Logistic Regression algorithm.

| | | No | | | | | | | |
|---|---|---|---|---|---|---|---|---|---|
| | | Hydration | 0.5 L | 0.75 L | 1 L | 1.25 L | 1.5 L | 1.75 L | 2 L |
| No | | 11 | 0 | 0 | 0 | 0 | 0 | 0 | 0 |
| Hydration | | | | | | | | | |
| 0.5 | L | 0 | 11 | 0 | 0 | 0 | 0 | 0 | 0 |
| 0.75 | L | 0 | 0 | 11 | 0 | 0 | 0 | 0 | 0 |
| 1 | L | 0 | 0 | 0 | 11 | 0 | 0 | 0 | 0 |
| 1.25 | L | 0 | 0 | 0 | 0 | 11 | 0 | 0 | 0 |
| 1.5 | L | 0 | 0 | 0 | 0 | 0 | 11 | 0 | 0 |
| 1.75 | L | 0 | 0 | 0 | 0 | 0 | 0 | 11 | 0 |
| 2 | L | 0 | 0 | 0 | 0 | 0 | 0 | 0 | 11 |

It can be seen from Table 2 that true positives (e.g. No Hydration-No Hydration, 0.5 L-0.5L etc., that are located in the diagonal of the table) outweigh any other prediction errors outside the diagonal. As such, the trained machine learning model based on the Logistic Regression algorithm can be considered as correctly predicting and classifying the correct level of fluid based on the measured S₁₁-parameter. It can be seen from Table 2 that there were also no misclassifications. This may be due to the Logistic Regression algorithm's ability to interpret differences amongst the radiofrequency sensors measurements between the different levels of water.

Table 3 shows a classification report, including a precision, recall, F1-score, and support score for each of the levels of water after the second data set was passed to the trained machine learning model based on the Logistic Regression algorithm.

| | Precision | Recall | F1 Scores | Support |
|---|---|---|---|---|
| | (%) | (%) | (%) | (#) |
| No | 1 | 1 | 1 | 11 |
| Hydration | | | | |

| | | | | | |
|---|---|---|---|---|---|
| 0.5 | L | 1 | 1 | 1 | 11 |
| 0.75 | L | 1 | 1 | 1 | 11 |
| 1 | L | 1 | 1 | 1 | 11 |
| 1.25 | L | 1 | 1 | 1 | 11 |
| 1.5 | L | 1 | 1 | 1 | 11 |
| 1.75 | L | 1 | 1 | 1 | 11 |
| 2 | L | 1 | 1 | 1 | 11 |

The inventors have found that the trained machine learning model based on the Logistic Regression algorithm had an overall accuracy of 100% on the second data set. This may be expected due to the high performance of the trained machine learning model based on the Logistic Regression algorithm on the first dataset. Precision may be understood as an accuracy of positive predictions. From Table 3, it can be seen that each level of water was predicted with a precision of 100%. This may be due to all the predictions being correct based on the data provided. Recall may be understood as a percentage of positive cases that were found by the classifier of the machine learning model based on the Logistic Regression algorithm. It can be seen from Table 3 that this was also 100% for all of the levels of water. The F1 score is a weighted harmonic mean of the precision and recall. It can be understood as determining a percentage of positive predictions that are correct. The F1 score was determined as 100%. Support can be understood as a number of actual occurrence of a level of water in the second data set. It can be indicative of a number of data points for each level of water that was present in the second data set.

The use of the Logistic Regression algorithm may facilitate the training and testing of the machine learning model without requiring higher computational power.

The above description refers to the control system 8, e.g. the device 8b, as being configured to perform a number of steps. It will be appreciated that the steps described above may be part of a method of determining a fluid level and/or a composition of fluid, which will be described below, and that the processor 8c of the processing device 8b may be configured to perform one or more of the steps. Additionally or alternatively, it will be appreciated that the terms “reflection coefficient”, “transmission coefficient”, “pre-determined reflection coefficient” and/or “pre-determined transmission coefficient” may be considered as encompassing the terms “data associated with” or “data representing” any of these coefficients. These terms may be interchangeably used throughout the description.

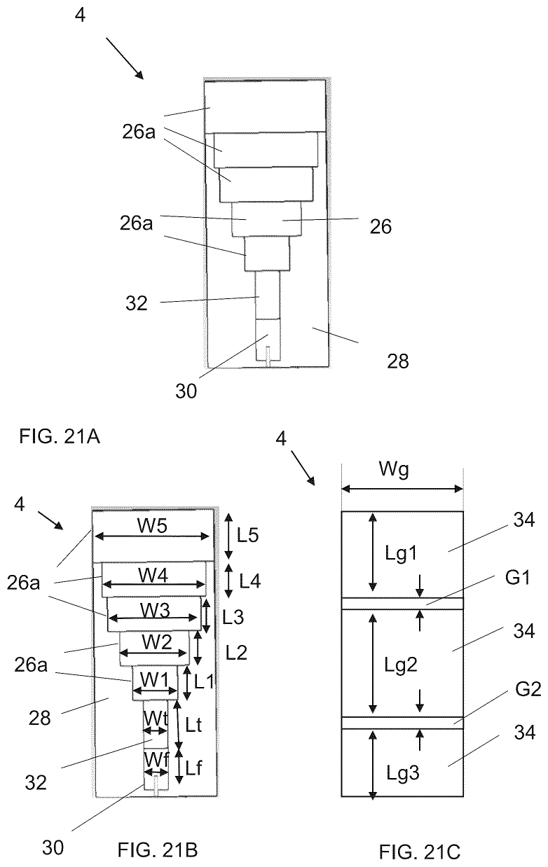

FIGS. 21A to 21C show an exemplary embodiment of a radiofrequency sensor 4 for use in a fluid level and/or fluid composition monitoring system, such as one or more of the systems 2 described above. FIGS. 21A and 21B show a plan view of the radiofrequency sensor 4. FIG. 21C shows an underneath view of the radiofrequency sensor 4. The radiofrequency sensor 4 may be provided in the form of a radiofrequency antenna. In the embodiment shown in FIGS. 21A to 21C, the radiofrequency sensor 4 is provided in the form of a stepped monopole antenna. The radiofrequency sensor 4 may comprise a conductive element 26. The conductive element 26 may be formed from a flexible or bendable conductive material. In this embodiment, the flexible or bendable conductive material comprises a conductive textile material, such as Shieldex® Zell or the like. A thickness of the conductive element 26 may be between about 0.05 mm and 0.5 mm, such as 0.1 mm.

The radiofrequency sensor 4 may comprise a substrate 28. The substrate 28 may be a flexible or bendable substrate 28. The substrate 28 may be formed from a flexible or bendable material, such as a textile material. By providing the radiofrequency sensor 4 with a flexible or bendable conductive element 26 and a flexible or bendable substrate 28, the radiofrequency sensor 4 may be unobtrusively included in the item of furnishing. In other words, this may allow for the radiofrequency sensor 4 not to be felt by the patient using the item of furnishing. For example, the flexible or bendable conductive element 26 and the flexible or bendable substrate 28 of the radiofrequency sensor 4 may allow for unobtrusive inclusion of the radiofrequency sensor 4 in an item of furnishing, such as a flexible or bendable item of furnishing. The flexible or bendable item of furnishing may comprise a mattress, sheet, duvet cover, pillow, curtain or the like. The flexible or bendable item of furnishing may comprise or be formed from a textile material or other flexible or bendable material. It will be appreciated that in other embodiments, the substrate and/or the conductive element may be rigid.

In this embodiment, the textile material comprises felt, such as viscose wool felt or the like. One or more electrical properties of the substrate 28 may comprise a relative permittivity and a loss tangent. For example, the substrate 28 may comprise a relative permittivity of about 1.45 to 1.55 and a loss tangent of about 0.02 to 0.068. A thickness of the substrate may be between about 2 mm to 10 mm, such as about 6 mm.

The radiofrequency sensor 4 may comprise a connection element 30. The connection element 30 may be part of or comprised in the conductive element 26. The connection element 30 may be provided in the form of a feed line or feeder. The connection element 30 may be configured to connect the radiofrequency sensor 4 to the control system 8, e.g. the controller 8a, e.g. using another connection element (not shown), such as a coaxial radiofrequency connector, coaxial cable and/or the like. A size or dimension of the connection element 30 may comprise a width Wf and a length Lf, which are indicated in FIG. 21B. For example, the length of the connection element 30 may be selected to match an impedance of about 50 Ohm of the other connecting element.

As can be seen in FIGS. 21A and 21B, the conductive element 26 comprises a plurality of sections 26a. In this embodiment, the conductive element 26 comprises five section 26a. It will be appreciated that in other embodiments, the conductive element 26 may comprise more or less than five sections.

Each of the sections 26 comprise a rectangular shape. A dimension or size of each section 26a may comprise a width W1 to W5 and a length L1 to L5. The width W1 to W5 of each of the sections 26 may be selected to reduce in towards the connection element 30.

The radiofrequency sensor 4 may comprise an impedance matching element 32. The impedance matching element 32 may be part of or comprised in the conductive element 26. The impedance matching element 32 may be arranged between the section 26 of the conductive element having the smallest width W1 and the connection element 30. The impedance matching element 32 may be configured to match an impedance of the connection element 30 with that of the sections 26a of the conductive element 26. For example, the impedance between the connection element 30 and the sections 26a of the conductive element 26 may be matched by varying a width Wt of the impedance matching element 32. The impedance matching element 32 may be provided in the form of a quarter wave transmission line. A dimension or size of the impedance matching element 32 may comprise the width Wt and a length Lt.

The radiofrequency sensor 4 may comprise one or more grounding elements 34, three of which are shown in FIG. 21C. The substrate 28 may be arranged between the conductive element 26 and the grounding elements 34. The grounding elements 34 may be formed from the same material as the conductive element 26. The grounding elements 34 may be arranged such that a gap G1, G2 is formed between two adjacent grounding elements 34. A dimension or size of each grounding element may comprise a width Wg and a length Lg1 to Lg3. The width Wg may be the same as the width W5 of the conductive element 26 having the largest width.

Exemplary values of the widths and length of each of the parts of the radiofrequency sensor 4 shown in FIGS. 21A to 21C are summarised in the table below. It will be appreciated that in other embodiments the radiofrequency sensor may be configured differently. For example, in such other embodiments, the radiofrequency sensor or one or more parts thereof may comprise a different shape and/or a different dimension or size than that disclosed herein.

| Dimension | Value (mm) |
|---|---|
| W5 | 35 |
| L5 | 15 |
| W4 | 30 |
| L4 | 10 |
| W3 | 27 |
| L3 | 10 |
| W2 | 20 |
| L2 | 10 |
| W1 | 13 |
| L1 | 10 |
| Wt | 7 |
| Lt | 14 |
| Wf | 17 |
| Lf | 12 |
| G1 | 3 |
| G2 | 2 |
| Lg1 | 25 |
| Lg2 | 30 |
| Lg3 | 25 |

The radiofrequency sensor 4 may be configured such as to have an operating frequency in the range of about 1.5 GHz to 4 GHz. However, it will be appreciated that in other embodiments, the radiofrequency sensor may have an operating frequency between about 1 GHz and 50 GHz or between about 1 GHz and 100 GHz.

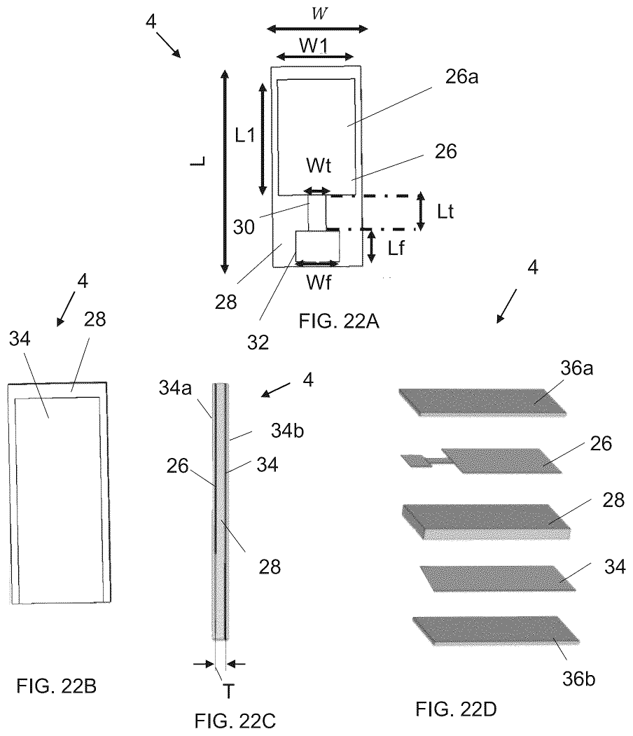

FIGS. 22A to 22D show another exemplary embodiment of a radiofrequency sensor 4 for use in a fluid level and/or fluid composition monitoring system, such as one or more of the systems 2 described above. FIG. 22A shows a plan view of the radiofrequency sensor 4. FIG. 22B shows an underneath view of the radiofrequency sensor 4. FIG. 22C shows a side view of the radiofrequency sensor 4. FIG. 22D shows an exploded view of the radiofrequency sensor 4. The radiofrequency sensor 4 shown in FIGS. 22A to 22D is similar to the radiofrequency sensor 4 shown in FIGS. 21A to 21C. Any of the features described above in relation to the radiofrequency sensor shown in FIGS. 21A to 21C may also apply to the radiofrequency sensor shown in FIGS. 22A to 22D. Only differences will be described in the following.

In the embodiment shown in FIGS. 22A to 22D, the conductive element 26 comprises a single section 26a. A width and a length of the section 26a are indicated in FIG. 22A by reference numerals W1 and L1, respectively. The radiofrequency sensor 4 may be provided in the form of a rectangular monopole antenna.

In this embodiment, the substrate 28 may be formed from a flexible or bendable material, such as an elastomeric material. The elastomeric material may comprise a polymer material, such as a rubber material, silicone rubber or the like. For example, the elastomeric material may be provided in the form of a Tap Plastic Platinum Silicone Rubber. The substrate 28 may comprise a relative permittivity of about 2.99 to 3.67 and a loss tangent of about 0.032. A thickness T of the substrate may be between about 2 mm to 10 mm, such as about 3 mm, and is indicated in FIG. 22C.

As described above, the radiofrequency sensor 4 may comprise a connection element 30 and an impedance matching element 32. However, the dimension of the section 26a of the conductive element 26, the connection element and the impedance matching element may be different due to the different electrical properties of the substrate 28.

In this embodiment, the radiofrequency sensor 4 may comprise a single grounding element 34. As described above, the substrate 28 may be arranged between the conductive element 26 and the grounding element 34. In this embodiment, the radiofrequency sensor 4 may comprise a first cover portion 36a and a second cover portion 36b. The first and second cover portions 36a, 36b may be formed from the same material as the substrate 28. It will be appreciated that in other embodiments, the first and second cover portions may be formed from a different material as the substrate. The first and second cover portions 36a, 36b may be configured to encase the conductive element 26, the substrate 28 and the grounding element 34.

A dimension or size of the first and second cover portions 36a, 36b may comprise a width W and a length L. By providing the radiofrequency sensor 4 with first and second cover portions 36a, 36b, the conductive element 26 and/or grounding element 34 may be protected from contamination, fluids or like and/or washing of item of furnishing or other item including the radiofrequency sensor 4 may be possible.

Exemplary values of the widths and length of each of the parts of the radiofrequency sensor 4 shown in FIGS. 22A to 22D are summarised in the table below. It will be appreciated that in other embodiments the radiofrequency sensor may be configured differently. For example, in such other embodiments, the radiofrequency sensor or one or more parts thereof may comprise a different shape and/or a different dimension or size than that disclosed herein.

| Dimension | Value (mm) |
|---|---|
| W1 | 30 |
| L1 | 50 |
| Wt | 7 |
| Lt | 13 |
| Wf | 17 |
| Lf | 12 |
| Lg | 75 |
| Wg | 30 |
| W | 35 |
| L | 75 |

The radiofrequency sensor 4 may be configured such as to have an operating frequency in the range of about 1.08 GHz to 4.13 GHz. However, it will be appreciated that in other embodiments, the radiofrequency sensor may have an operating frequency between about 1 GHz and 50 GHz or between about 1 GHz and 100 GHz.

The radiofrequency sensor 4 shown in FIGS. 22A to 22D may be formed by moulding and curing the second cover portion 36b from the elastomeric material. The grounding element 34 may be arranged on the cured second portion 36b. The substrate 28 may be formed on the grounding element 34. For example, the elastomeric material may be liquid and may be poured on the grounding element 34 to cover the grounding element 34. This may result in the grounding element 34 being encapsulated by the elastomeric material and/or forming the substrate 28, when the elastomeric material is cured. The conductive element 26 may be arranged on the substrate 28. The first cover portion 36a may be formed on the conductive element 26. For example, the elastomeric material may be liquid and may be poured on the conductive element 26 to cover the conductive element 26. This may result in the conductive element 32 being encapsulated by the elastomeric material and/or the first cover portion 36a being formed, once the elastomeric material is cured. Another connecting element (not shown), such as a coaxial radiofrequency connector, may be connected to each of the conductive element 26 and the grounding element 34, e.g. using an epoxy material, such as silver epoxy. Each of the first and second cover portions 36a, 36b may comprise a thickness between 0.5 mm and 3 mm, such as 1 mm.

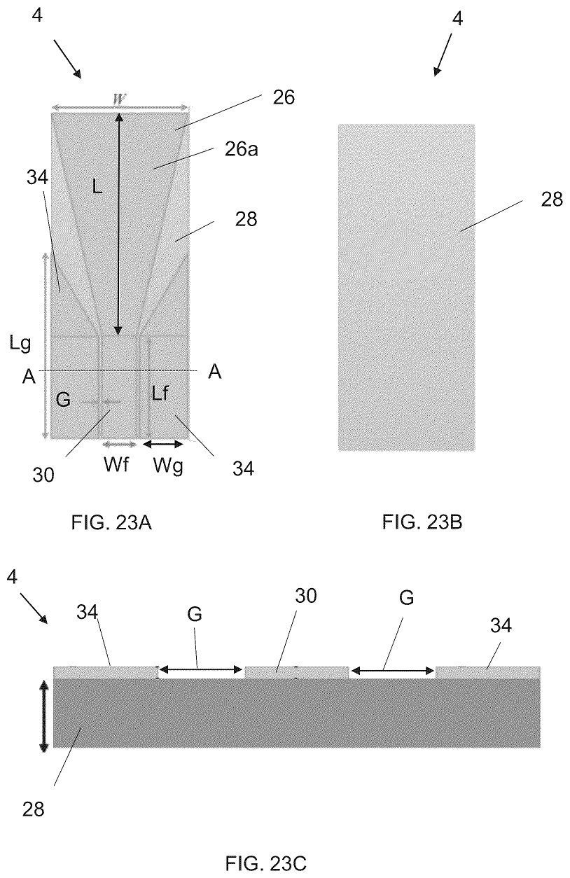

FIGS. 23A to 23C show another exemplary embodiment of a radiofrequency sensor 4 for use in a fluid level and/or fluid composition monitoring system, such as one or more of the systems 2 described above. FIG. 23A shows a plan view of the radiofrequency sensor 4. FIG. 23B shows an underneath view of the radiofrequency sensor 4. FIG. 23C shows a sectional view of the radiofrequency sensor 4 along line A-A′ in FIG. 23A.

The radiofrequency sensor 4 shown in FIGS. 23A to 23C is similar to the radiofrequency sensor 4 shown in FIGS. 21A to 21C and/or FIGS. 22A to 22D. Any of the features described above in relation to the radiofrequency sensor shown in FIGS. 21A to 21C and/or FIGS. 22A to 22D may also apply to the radiofrequency sensor shown in FIGS. 23A to 23C. Only differences will be described in the following.

In this embodiment, the radiofrequency sensor 4 is provided in the form of a trapezoidal monopole antenna. In this embodiment, the conductive element 26 comprises a single section 26a. The section 26a of the conductive element 26 may comprise a trapezoidal shape. A dimension or size of the section 26a may comprise a width W and a length L.

The conductive element 26 may be formed on the substrate 28 using a lithographic method, such as photolithography or the like. The conductive element 26 may be formed from a metal material, such as copper, aluminium or the like, or a transition metal material, such as silver, gold or the like. The substrate 28 may comprise a composite material, such as a glass-reinforced epoxy material. For example, the substrate 28 may comprise a FR-4 substrate. The substrate 28 may comprise a relative permittivity of about 4.4 and a loss tangent of about 0.02 to 0.03. A thickness of the substrate 28 may be selected such that the substrate 28 and the conductive element 26 formed thereon are bendable. For example, a thickness of the substrate 28 may be between 0.1 mm and 0.5 mm, such as 0.2 mm. However, it will be appreciated that in other embodiments, a thickness of the substrate may be selected such that the substrate and/or the conductive element may be rigid. In such other embodiments, the radiofrequency sensor may be included in a rigid part of the item of furnishing, such as a headboard of a bed, a base plate of a chair or the like, or the other item.

In this embodiment, the impedance matching element 32 may be integral with the connection element 30. As such, the following description will refer to the connection element 30 only. However, it will be appreciated that an impedance of the connection element 30 may be matched to the section 26a of the section 26a of the conductive element 26. The connection element 30 may be provided in the form of a co-planar waveguide transmission line. A dimension or size of the connection element 30 may comprise a width Wf and a length Lf.

In this embodiment, the radiofrequency sensor 4 comprises two grounding elements 34. A dimension of size of each grounding element 34 may comprise a length Lg and a width Wg. The length Lg of each grounding element 34 may be understood as the length of the longest side of each grounding element 34. The grounding elements 34 may be arranged on the same plane as the conductive element 26. The grounding elements 34 may be arranged on either side of the connection element 30. Each of the grounding elements 34 may have a trapezoidal shape. The grounding element 34 and the conductive element 26 may be arranged such that a gap G is formed between the connection element 30 and each grounding element 34.

Exemplary values of the widths and length of each of the parts of the radiofrequency sensor 4 shown in FIGS. 23A to 23C are summarised in the table below. It will be appreciated that in other embodiments the radiofrequency sensor may be configured differently. For example, in such other embodiments, the radiofrequency sensor or one or more parts thereof may comprise a different shape and/or a different dimension or size than that disclosed herein.

| Dimension | Value (mm) |
|---|---|
| W | 30 |
| L | 50 |
| G | 0.5 |
| Wf | 5 |
| Lf | 20 |
| Wg | 12 |
| Lg | 44 |

The radiofrequency sensor 4 may be configured such as to have an operating frequency in the range of about 1.5 GHz to 4 GHz. However, it will be appreciated that in other embodiments, the radiofrequency sensor may have an operating frequency between about 1 GHz and 50 GHz or between about 1 GHz and 100 GHz.

Any of the radiofrequency sensors 4 described above in relation to FIGS. 21A to 23C may be part of or comprised in any of the exemplary systems 2 disclosed herein, e.g. in any combination. It will be appreciated that in other embodiments, one or more different radiofrequency sensors, e.g. one or more radiofrequency sensors having a different configuration, size and/or shape, may be part of or comprised in the any of the systems disclosed herein.

A specific absorption rate was measured for the exemplary radiofrequency sensors 4 disclosed herein. The specific absorption rate may be understood as a measure how electro-magnetic waves are absorbed by tissue of a human body. The maximum limit of the specific absorption rate imposed by the government in the United Kingdom is 2 W/kg for 10 g of tissue mass. The specific absorption rate for each of the exemplary radiofrequency sensors 4 disclosed herein was measured to be below the imposed maximum limit for transmitting powers of each of the radiofrequency sensors 4 of 1 mW, 10 mW and 100 mW. For example, the specific absorption rate for each of the exemplary radiofrequency sensors 4 disclosed herein was measured to be below 0.4 W/kg for transmitting powers of each of the radiofrequency sensors 4 of 1 mW, 10 mW and 100 mW.

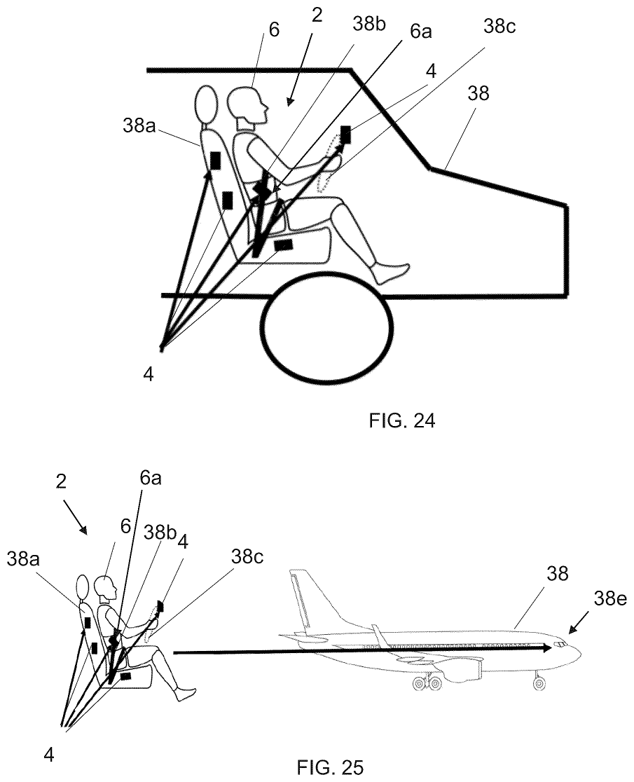

FIG. 24 shows an exemplary vehicle 38 comprising a fluid level and/or fluid composition monitoring system 2. Only the radiofrequency sensors, which are indicated by reference numeral 4 in FIG. 24, of the system 2 are shown for sake of clarity. However, it will be appreciated that the system 2 shown in FIG. 24 may comprise any of the features of any of the systems 2 described above. The following description refers to a person or subject. However, it will be appreciated that the system disclosed herein may additionally or alternatively be used for determining and/or monitoring a level of fluid and/or a composition of fluid of a part of a patient's body, as described above.

In some embodiments, the vehicle 38 is an automotive vehicle, such a vehicle for carrying one or more passengers, heavy loads of the like. For example, the vehicle 38 may comprise a car, lorry or the like. At least one radiofrequency sensor 4 is configured to be included in an item or part of the vehicle 38. The item or part of the vehicle 38 may comprise at least one of: a part of the vehicle 38 configured to support the person or subject or a part of the person or subject's body, such as a seat, headrest, armrest or the like, a fastening apparatus, such as a seat belt or one or more straps, a steering wheel, a door, a dashboard or other compartment of the vehicle, a wall and/or an item of furnishing or accessory of the vehicle.

In the embodiment shown in FIG. 24, a plurality of radiofrequency sensors 4 are included in the seat 38a, at least one radiofrequency sensor 4 is included in the seat belt 38b and at least one radiofrequency sensor 4 is included in the steering wheel 38c of the vehicle 38. However, it will be appreciated that in other embodiments, one or more radiofrequency sensors may be additionally or alternatively be included in other parts of the vehicle, such as a door, a part of the vehicle configured to support a part of a subject or person's body, such as an armrest, headrest or the like, a dashboard or other compartment of the vehicle, an item of furnishing or accessory of the vehicle and/or any other part of the vehicle that is in proximity to and/or surrounds the part of the person or subject's body to be monitored.

In this embodiment, the subject or person may be a driver of the vehicle 38. The system 2 may be included in the vehicle 38 to determine a level of fluid, a change of the level of fluid, a composition of fluid and/or a change of the composition of fluid in a part of the driver's body 6. The determined level of fluid and/or the change thereof in the part of the driver's body 6 may be indicative of a level of hydration, a state of health, a need, a condition, disease and/or medical condition of the driver. For example, the determined level of fluid and/or the change thereof may be indicative of the driver needing a break, rest and/or medical assistance. As described above, the system 2, e.g. the control system, may be configured to contact an emergency service, e.g. to request an ambulance or doctor, e.g. depending on the determined level of fluid and/or the change of the level of fluid in the part of the driver's body.

The determined composition of fluid and/or change thereof may be indicative of the presence of another fluid, such as alcohol. For example, the system 2 may be included in the vehicle 38 to determine a presence of alcohol, e.g. by monitoring the composition of fluid, a level of alcohol a change thereof in the part 6a of the driver's body 6 to be monitored. The part 6a of the driver's body 6 to be monitored comprises one or more organs of the driver, such as the stomach, kidneys and liver of the driver. As described above, the electrical properties of the organs and/or tissue may include the relative conductivity, the electrical resistivity and/or dielectric constant. The electrical properties of alcohol are different to the electrical properties of water. For example, an electrical resistivity of alcohol is lower than an electrical resistivity of water. As such, less of the first signal 10 may be absorbed by the part 6a of the driver's body 6 and at least a part of the determined reflection coefficient and/or transmission coefficient may be lower compared to embodiments where the fluid comprises only water. As such, the composition of fluid in the part 6a of the driver's body 6 can be determined, e.g. as described above, and/or the different fluids in the part 6a of the driver's body 6 can be distinguished.

The system 2, e.g. the control system 8, may be configured to monitor the composition of fluid and/or the level of alcohol in the part of the driver's body 6 and/or to report the determined level of alcohol or change thereof, e.g. to law enforcement or any other official organisation. It will be appreciated that in other embodiments, the person or subject may be a passenger of the vehicle.

It will be appreciated that in other embodiments, the vehicle shown in FIG. 24 may comprise a military vehicle, such as a tank, armoured vehicle, military aircraft or the like. The system may be included in the military vehicle to determine a level of fluid, a composition of fluid and/or a change thereof in the part of the subject or person's body. The determined level of fluid, composition of fluid and/or the change thereof in the part of the subject or person's body may be indicative of a level of hydration, a state of health, a condition, a need, a disease and/or a medical condition of the person or subject. For example, the determined level of fluid, composition of fluid and/or the change thereof may be indicative of the subject or person needing medical assistance. For example, the determined level of fluid, composition of fluid and/or change thereof may be indicative of an injury or wound, such as an internal or undetected injury or wound of the part of the subject or person's body.

A reflection absorption coefficient and/or transmission coefficient that has been determined by the system, as described above, may be indicative of a substance, e.g. a toxic, chemical and/or radioactive substance, in the part of the person or subject's body. For example, the determined composition of fluid may be indicative of the substance. The presence of the substance may cause a change in the electrical properties of the organs and/or tissue in the part of the subject or person's body to be monitored. This may lead to one or more parts or all of the determined reflection coefficient and/or transmission coefficient to be different, e.g. relative the one or more pre-determined reflection coefficient and/or transmission coefficient. This difference of the determined reflection coefficient and/or transmission coefficient may be indicative of the presence of the substance and/or a concentration, e.g. a relative concentration, of the substance in the part of the subject or person's body.

FIG. 25 shows another exemplary vehicle comprising a fluid level and/or fluid composition monitoring system 2. In this embodiment, the vehicle is an aircraft 38, such an aeroplane, helicopter or the like. It will be appreciated that any features described above in relation to FIG. 24 may also apply to the embodiment shown in FIG. 25. For example, in the embodiment shown in FIG. 25, a plurality of radiofrequency sensors 4 are included in a seat 38a, at least one radiofrequency sensor 4 is included in the seat belt 38b and at least one radiofrequency sensor 4 is included in the steering wheel 38c of the aircraft 38. However, it will be appreciated that in other embodiments, one or more radiofrequency sensors may be additionally or alternatively be included in other parts of the aircraft, such as a door, a part of the aircraft configured to support a part of a subject or person's body, such as an armrest, headrest or the like, a dashboard or other compartment of a cockpit 38e of the aircraft, an item of furnishing or accessory of the cockpit and/or any other part of the aircraft that is in proximity to and/or surrounds the part of the person or subject's body to be monitored.

In this embodiment, the subject or person may be a pilot of the aircraft 38. The system 2 may be included in the aircraft 38 to determine a level of fluid, a composition of fluid and/or a change thereof in a part of the pilot's body 6. The determined level of fluid, composition of fluid and/or the change thereof in the part of the pilot's body 6 may be indicative of a level of hydration, a state of health, a condition, a need, a disease and/or a medical condition of the pilot. For example, the determined level of fluid and/or the change thereof may be indicative of the pilot needing a break, rest and/or medical assistance.

Although FIGS. 24 and 20 show embodiments where the system 2 is included in an automotive vehicle and an aircraft, it will be appreciated that the system disclosed herein is not limited to these uses or applications. For example, in other embodiments, the vehicle may be a spacecraft, a space station or the like.

In embodiments where the vehicle is a spacecraft or space station, one or more radiofrequency sensors may be included in any part of the spacecraft or space station. In such embodiments, the subject or person may be an astronaut. The one or more radiofrequency sensors may be included in an item of furnishing, a part of a cockpit or any other part of the spacecraft and/or space station that is in proximity to and/or surrounds the part of the astronaut's body to be monitored.

The system may be configured to determine a level of fluid, composition of fluid and/or change thereof in part of the astronaut's body. For example, the part of the astronaut to be monitored may comprise the stomach of the astronaut. In this embodiment, the fluid may comprise water, liquid food and/or semisolid food. The determined level of fluid, composition of fluid and/or the change thereof in the part of the astronaut's body 6 may be indicative of a level of hydration and/or a level of food. However, it will be appreciated that in other embodiments, the part of the astronaut to be monitored may comprise additionally or alternatively other organs and/or tissue of the astronaut's body. As such, the determined level of fluid, composition of fluid and/or the change thereof in the part of the astronaut's body may be additionally or alternatively indicative of a state of health, a condition, a need, a disease and/or a medical condition of the astronaut.

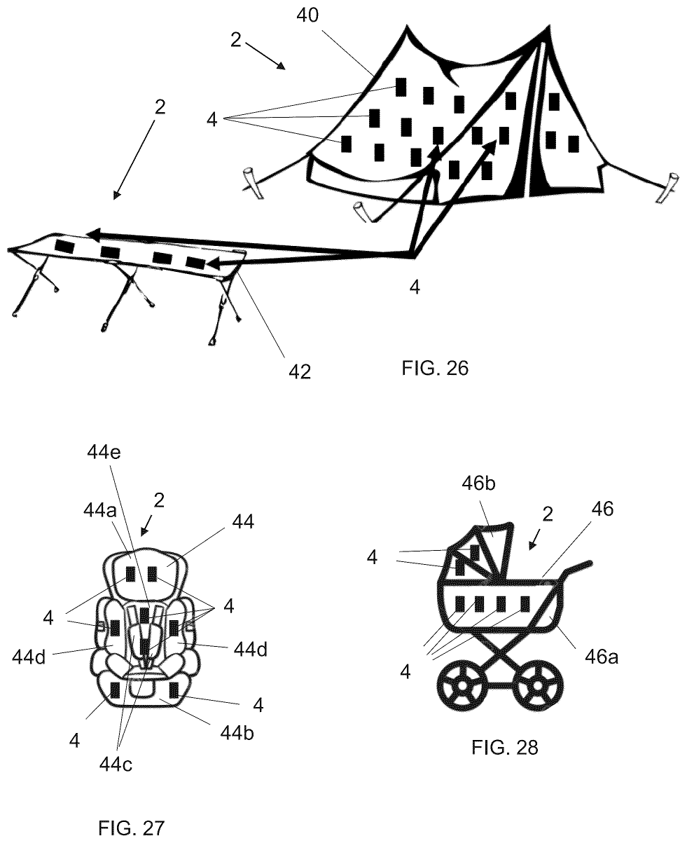

FIG. 26 shows an exemplary shelter or dwelling 40 and an item 42 of furnishing of the shelter or dwelling 40 comprising a fluid level and/or fluid composition monitoring system 2. The shelter or dwelling 40 and the item 42 of furnishing may also be considered as an outdoor or sports item, which will be described below. Only the radiofrequency sensors, which are indicated by reference numeral 4 in FIG. 26, of the system 2 are shown for sake of clarity. However, it will be appreciated that the system 2 shown in FIG. 26 may comprise any of the features of any of the systems 2 described above. In this embodiment, the shelter or dwelling 40 is provided in the form of a collapsible and/or temporary shelter, such as a tent. However, it will be appreciated that in other embodiments, the shelter or dwelling may be provided in a different form, such as a cabin or the like.

The item 42 of furnishing of the shelter or dwelling 40 may comprise an item for supporting a person or subject's body. For example, the item 42 of furnishing of the shelter or dwelling 40 may comprise an item of furniture, such as a foldable item of furniture. In the embodiment shown in FIG. 26, the item of furnishing 42 may comprise a camping bed. However, it will be appreciated that in other embodiments, the item of furnishing may comprise a camping chair or other items of furnishing, such as a sleeping bag, blanket or the like.

At least one radiofrequency sensors 4 may be included in the shelter or dwelling 40 and/or the item 42 of furnishing of the shelter or dwelling 40. A first plurality of radiofrequency sensors 4 may be included in the shelter or dwelling 40. For example, the first plurality of radiofrequency sensors 4 may be included in one or more walls 40a of the shelter or dwelling 40. A second plurality of sensors 4 may be included in the item of furnishing 42. The first and/or second plurality of radiofrequency sensors 4 may be arranged to be in proximity to the part of the person or subject's body to be monitored (not shown in FIG. 26).

The system or a part thereof may be included in the shelter or dwelling 40 and/or the item 42 of furnishing thereof to determine a level of fluid, a composition of fluid and/or a change thereof in a part of the subject or person's body. The determined level of fluid, composition of fluid and/or the change thereof in the part of the subject or person's body may be indicative of a state of health, a level of hydration, a condition, a need, a disease and/or medical condition. For example, the determined level of fluid, composition of fluid and/or the change thereof may be indicative of the subject or person needing medical assistance. The determined level of fluid, composition of fluid and/or change thereof may be indicative of an injury or wound, such as an internal or undetected injury or wound of the part of the person or subject's body. It will be appreciated that the terms “item of furnishing,” as described above, encompass the terms “item of furnishing of a shelter or dwelling.”

FIG. 27 shows an exemplary item comprising a fluid level and/or fluid composition monitoring system 2. Only the radiofrequency sensors, which are indicated by reference numeral 4 in FIG. 27, of the system 2 are shown for sake of clarity. However, it will be appreciated that the system 2 shown in FIG. 27 may comprise any of the features of any of the systems 2 described above. The item may comprise an item associated with a vehicle. For example, in the embodiment shown in FIG. 27, the item comprises an apparatus 44 configured to support and/or restrain a baby, infant or child, e.g. in a vehicle. For example, the apparatus 44 may be provided in the form of a seat or car seat or the like. In the embodiment shown in FIG. 27, one or more radiofrequency sensors 4 are included in one or more parts of the apparatus 44. The one or more parts of the apparatus 44 may comprise one or more parts configured to support a part of the baby, infant or child's body. For example, the one or more parts of the apparatus 44 comprise a headrest 44a, a leg rest 44b, one or more sides 44d and/or a backrest 44e. The one or more parts of the apparatus 44 may also comprise a fastening apparatus 44c, such as one or more straps.

In this embodiment, two radiofrequency sensors 4 are included in the headrest 44a, two radiofrequency sensors 4 are included in the leg rest 44b, at least one radiofrequency sensor 4 is included in the straps 44c and at least one radiofrequency sensor 4 is included in the backrest 44e. It will be appreciated that the apparatus is not limited to the arrangement of radiofrequency sensors shown in FIG. 27. For example, one or more radiofrequency sensors may be included in the apparatus in proximity to and/or on opposite sides of a part of a baby, child or infant's body to be monitored.

FIG. 28 shows another exemplary vehicle 46 comprising a fluid level and/or fluid composition monitoring system 2. Only the radiofrequency sensors, which are indicated by reference numeral 4 in FIG. 28, of the system 2 are shown for sake of clarity. However, it will be appreciated that the system 2 shown in FIG. 28 may comprise any of the features of any of the systems 2 described above. The vehicle 46 is provided in the form of a vehicle for transporting a baby, child or infant, such as a pram, baby, child or infant buggy or pushchair, or other vehicle. One or more radiofrequency sensors 4 may be included in one or more parts of the vehicle 46. The one or more parts of vehicle 46 may comprise a wall 46a, such as a sidewall, of the vehicle 46, one or more straps (not shown) and/or a canopy or hood 46b. It will be appreciated that in other embodiments, the one or more sensors may be included in another part of the vehicle, such as a mattress, a base, a cover and/or the like. It will be appreciated that the vehicle is not limited to the arrangement of radiofrequency sensors shown in FIG. 28. For example, one or more radiofrequency sensors may be included in the vehicle in proximity to and/or on opposite sides of a part of a baby, child or infant's body to be monitored.

In the embodiments shown in FIGS. 27 and 28, the subject or person may be a baby, child or infant. The system 2 may be included in the item 44 to determine a level of fluid, a composition of fluid and/or a change thereof in a part of the baby, child or infant's body. The part of the baby, child or infant's body may comprise one or more organs, such as the stomach, kidneys and/or bladder of the baby or infant. The determined level of fluid, composition of fluid and/or the change thereof in the part of the baby, child or infant's body 6 may be indicative of a state of health, a condition, a level of hydration, a level of food, a disease and/or a medical condition of the baby or infant.

For example, the fluid may comprise liquid flood, semisolid food and/or other food. In such examples, the determined level of fluid, composition of fluid and/or change thereof may be indicative of whether the baby, child or infant needs more food, e.g. liquid food, semisolid food or other food, or is well fed, e.g. when travelling in a vehicle, such as automotive vehicle, an aircraft or the like. This may allow for a prediction of time when the baby, child or infant needs to be fed.

Additionally or alternatively, the fluid may comprise water. In such examples, the determined level of fluid, composition of fluid and/or change thereof may be indicative of a level of fluid, composition of fluid and/or change thereof in an organ of the baby, child or infant, e.g. the bladder and/or kidneys. This may allow for a prediction of time when a nappy of the baby, child or infant needs to be changes, bed-wetting, other accidental urination or the like.

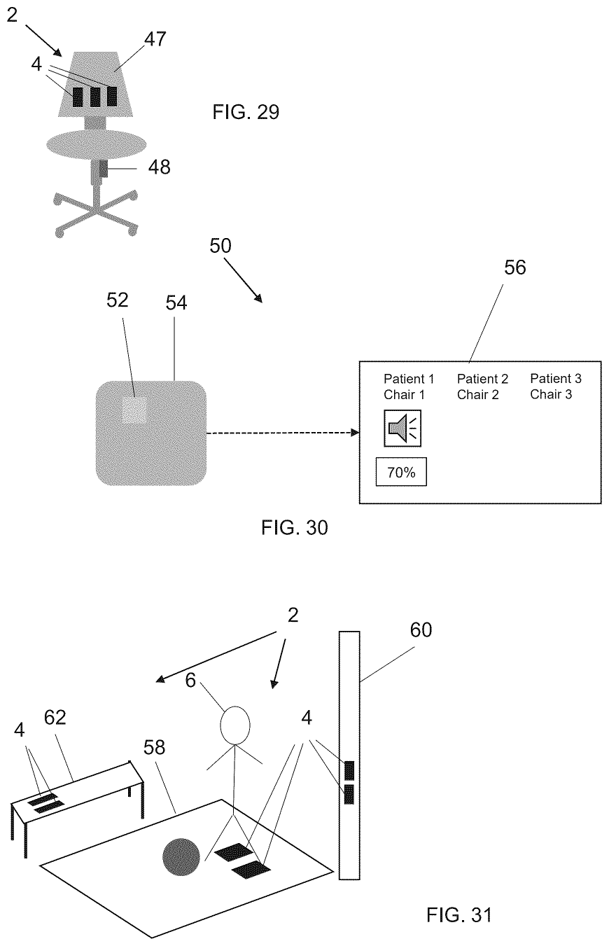

FIG. 29 shows another exemplary item of furnishing comprising a fluid level and/or fluid composition monitoring system 2. Only the radiofrequency sensors, which are indicated by reference numeral 4 in FIG. 29, of the system 2 are shown for sake of clarity. However, it will be appreciated that the system 2 shown in FIG. 29 may comprise any of the features of any of the systems 2 described above.

In the embodiment shown in FIG. 29, the item of furnishing is provided in the form of chair 47, such as an office chair, a chair used in a factory or manufacturing facility or the like. As mentioned above, the system 2 described herein may be used for monitoring a level of fluid and/or a composition of fluid in a part of a body of a subject or person in a business environment or work environment, such as an office or the like, factory environment, manufacturing environment or the like. The system 2 may be included in the chair 47 to determine a level of fluid, a composition of fluid and/or a change thereof in a part of the subject or person's body. The part of the subject or person's body may comprise one or more organs, such as the stomach, kidneys and/or bladder of the baby or infant. The determined level of fluid, composition of fluid and/or the change thereof in the part of the subject or person's body may be indicative of a state of health, a condition, a level of hydration, a level of food, a disease and/or a medical condition of the subject or person. This may allow for the health of staff or employees to be monitored.

The system 2 may be used on its own or in combination with one or more other sensors. For example, the system 2 may be used in combination with a pressure or weight sensor 48. The pressure or weight sensor 48 may be configured to sense a pressure exerted on or weight applied to the chair 47.

Although FIG. 29 shows the radiofrequency sensor 4 as being included in a chair, it will be appreciated that in other embodiments, the radiofrequency sensor may be included in another item of furnishing in the business environment, work environment, factory environment, manufacturing environment or the like.

FIG. 30 schematically shows an exemplary alert system 50 for use in or with the system 2. The alert system 50 may be part of the control system 8. As described above, depending on the determined level of fluid, the change of the level of fluid, the composition of fluid and/or the change of composition of fluid in the part of the patient's body, the control system 8 may be configured to alert a user of the system 2, such as a doctor, carer, parent, law enforcement or other health care professional. In some embodiments, the alert may be or comprise an alarm, an alert message, an automated alert or the like.

In the embodiment shown in FIG. 30, the alert system 50 comprises an alarm device 52. The alarm device 52 may be implemented as an optical or visual alarm device or an acoustic alarm device. For example, when the alarm device 52 is implemented as the visual or optical alarm device, the alarm device may comprise a light source, such as LED or the like. When the alarm device 52 is implemented as the audio or acoustic alarm device, the alarm device may comprise an acoustic sound emitting device, such as a loudspeaker to the like.

The alarm device 52 may be configured to be included in an item of furnishing or another item 54, as described herein. For example, the alarm device 52 may be included in the same item or furnishing or other item as one or more of the radiofrequency sensors described herein. Alternatively, the alarm device 52 may be included in another item of furnishing or yet another item. In the embodiment shown in FIG. 30, the alarm device 52 is included in an item of furnishing 54, which is provided in the form of a cushion. The radiofrequency sensors have been omitted from FIG. 30 for sake of clarity.

The alarm device 52 may be configured to transmit a signal to the control system 8. The signal may be indicative of the alert. The signal may also be indicative of the determined level of fluid, the change of the level of fluid, the composition of fluid and/or the change of composition of fluid in the part of the patient's body. Additionally, the signal may be indicative of an identity of the patient and/or a location of the patient, for example in a room.

The alarm system 50 may comprise a screen 56, e.g., a CRT (cathode ray tube), plasma, LED (light emitting diode) or LCD (liquid crystal display) monitor, for displaying information to a user of the system 2. The screen 56 may be part of the control system 8, e.g. the device 8b. However, it will be appreciated that in some embodiments, the screen may be separately provided from the device 8b.

The information displayed on the screen may be indicative of the determined level of fluid, the change of the level of fluid, the composition of fluid and/or the change of composition of fluid in the part of the patient's body. Additionally, the information displayed on the screen 56 may be indicative of the identity of the patient and/or the location of the patient, for example in the room. The information displayed on the screen 56 may be displayed as part of a graphical data report, such as a dashboard or the like. In the example shown in FIG. 30, the alarm system 50 alerts a user that a fluid level of Patient 1, who is located in chair 1 in the room, is 70% and as such, this patient is likely to need to use the bathroom.

Although FIG. 30 shows a single alarm device 52, it will be appreciated that the alarm system 50 may comprise a plurality of alarm devices 52. Each of the plurality of alarm devices 52 may be associated with a respective patient, subject or person. Each of the alarm devices 52 may be configured to transmit a respective signal to the control system 8, as described above.

FIG. 31 shows an exemplary an outdoor or sports item 60 comprising the system 2 described herein. FIG. 31 also shows an exemplary outdoor or sports area comprising the system 2. Only the radiofrequency sensors, which are indicated by reference numeral 4 in FIG. 31, of the system 2 are shown for sake of clarity. However, it will be appreciated that the system 2 shown in FIG. 31 may comprise any of the features of any of the systems 2 described above.

The radiofrequency sensors 4 may be included, e.g. embedded or integrated, in the outdoor item or sports item and/or outdoor or sports area such that the system 2 can determine a level of fluid, a composition of fluid and/or a change thereof in a part of a subject or person's body 6. In the example shown in FIG. 31, the person or subject may be an athlete, a player or the like. The determined level of fluid, composition of fluid and/or the change thereof in the part of the subject or person's body may be indicative of a state of health, a level of hydration, a condition, a need, a disease and/or medical condition. For example, the determined level of fluid, composition of fluid and/or the change thereof may be indicative of the subject or person needing medical assistance. The determined level of fluid, composition of fluid and/or change thereof may be indicative of a low level of hydration.

The outdoor or sports item 60 or the outdoor or sports area may comprise an outdoor or sports item or outdoor or sports area that is located or locatable in proximity to the subject or person, e.g. in use. For example, the outdoor or sports item or the outdoor or sports area may be located or locatable relative to the person or subject such that the person or subject is within a range of the radiofrequency sensors 4, e.g. in use. The range of the radiofrequency sensors 4 may be between about 1 meter and about 2 meters. The outdoor or sports item or the outdoor or sports area may be arranged or arrangeable in proximity to a part of the body 6 of the subject or person to be monitored, e.g. in use. The outdoor or sports item 60 or the outdoor or sports area 58 may be arranged or arrangeable to separate from the subject or person.

The outdoor or sports item 60 may comprise outdoor or sports equipment. The outdoor or sports item may comprise as a post, goal post, a fence, a gate or the like. In the example shown In FIG. 31, the outdoor or sports area comprises a surface 58. The surface 58 may be implemented as a playing surface, such as a pitch or court, as a running surface, such as a running track, as a cycling surface, such as a cycling track, and/or the like. The radiofrequency sensors 4 may be included in the surface 58 of the outdoor or sports area. The sports area may comprise an indoor or outdoor sports area. It will be appreciated that the radiofrequency sensor may be included in other sports or outdoors items than those described above.

It will be appreciated that the item of furnishing described herein may comprise an item of furnishing for use in an outdoor or sports environment. The outdoor or sports environment may comprise a sports or outdoor facility. The sports or outdoor facility may also be referred to as a sports or outdoor venue. The sports or outdoor facility may comprise a medical area, a medical facility, a changing area or room, and/or the like. The item of furnishing may comprise an item of furniture. The item of furniture may comprise a seat, such as a bench, chair and/or the like, a bed or other item of furniture.

FIG. 31 also schematically shows an exemplary item of furnishing for use in the outdoor or sports environment. The item of furnishing comprises the system 2 described herein. In the example shown in FIG. 31, the item of furnishing is provided in the form of a bench 62. The radiofrequency sensors 4 are included, e.g. embedded or integrated, in the bench 62. The bench 62 may be provided for use in a substitute area, rest area, medical area, changing area or the like of the outdoor or sports environment.

The system 2 may be used to determine a level of fluid, a composition of fluid and/or a change thereof in a part of the subject or person's body 6. The determined level of fluid, composition of fluid and/or the change thereof in the part of the subject or person's body may be indicative of a state of health, a level of hydration, a condition, a need, a level of fatigue, a disease and/or medical condition. For example, the determined level of fluid, composition of fluid and/or the change thereof may be indicative the subject or person needing medical assistance. The determined level of fluid, composition of fluid and/or change thereof may be indicative of fatigue and/or a low level of hydration. By including the radiofrequency sensors 4 in the item of furnishing for use in the outdoors or sports environment, the level of fluid or hydration of the subject or person may be monitored before or after an outdoor or sports activity.

It will be appreciated that the outdoor or sports item or outdoor or sports area described above may also be part of or used in the outdoor or sports environment.

Although in each of the embodiments shown in FIGS. 24 to 29 and 31 a plurality of radiofrequency sensors 4 are shown, it will be appreciated that the system disclosed herein is not limited to the arrangement of radiofrequency sensors shown in each of FIGS. 24 to 29 and 31. The radiofrequency sensors shown in each of FIGS. 24 to 29 and 31 are intended to show a number of possible options of how one or more radiofrequency sensor or an array of radiofrequency sensors may be included in the exemplary items, areas, apparatuses or parts described herein. For example, in other embodiments, only a single radiofrequency sensor or a single array of radiofrequency sensors may be included in the items, areas, apparatuses or parts described in relation to any one of FIGS. 24 to 29 and 31. Additionally or alternatively, first and second radiofrequency sensors and/or first and second arrays of radiofrequency sensors may be included in one or more of the items, apparatuses or parts described in any one of FIGS. 24 to 29 and 31, e.g. such that the first and second radiofrequency sensors and/or the first and second arrays of radiofrequency sensors are on opposite sides of a part of the person or subject's body.

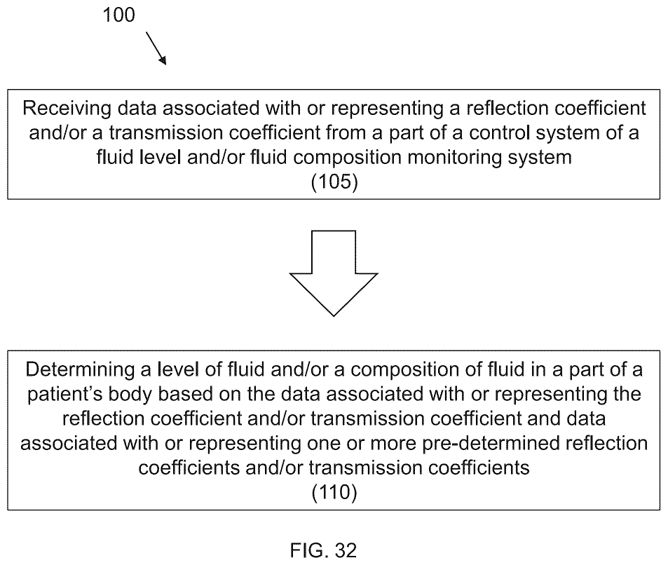

FIG. 32 shows an exemplary flow diagram outlining the steps of an exemplary method 100 of determining a level of fluid and/or a composition of fluid. In step 105, the method 100 comprises receiving data associated with or representing a reflection coefficient and/or a transmission coefficient from a part of a control system of a fluid level and/or fluid composition monitoring system. The fluid level and/or fluid composition monitoring system may comprise any features of the fluid level and/or fluid composition monitoring systems 2 described above. The part of the control system 8 may comprise the controller 8a. The reflection coefficient and/or the transmission coefficient may be determined by the controller 8a based on the first and second signals 10, 12, as described above.

In step 110, the method 100 comprises determining the level of fluid and/or the composition of fluid in a part of a patient's body based on the data associated with or representing the reflection coefficient and/or transmission coefficient and data associated with or representing the one or more pre-determined reflection coefficient and/or transmission coefficient described above.

The method 100 may comprise comparing the data associated with or representing the reflection coefficient and/or transmission coefficient to the data associated with or representing the one or more pre-determined reflections and/or transmission coefficients, respectively. The method 100 may comprise determining the level of fluid and/or the composition of fluid in the part 6a of the patient's body 6 based on the comparison between data associated with or representing the reflection coefficient and/or transmission coefficient and the data associated with or representing the one or more pre-determined reflection coefficients and/or transmission coefficients, respectively. The method 100 may comprise correlating one or more changes between the data associated with or representing the reflection coefficient and/or transmission coefficient and the data associated with or representing the one or more pre-determined reflection coefficients and/or transmission coefficients, respectively, to a level of fluid in the part 6a of the patient's body 6 and/or a change of the level of fluid in the part 6a of the patient's body 6. The method 100 may comprise correlating one or more changes between the data associated with or representing the reflection coefficient and/or transmission coefficient and the data associated with or representing the one or more pre-determined reflection coefficients and/or transmission coefficients, respectively, to a composition of fluid in the part 6a of the patient's body 6 and/or a change of the composition of fluid in the part 6a of the patient's body 6.

The method 100 may comprise determining the level of fluid, the change of the level of fluid, the composition of fluid and/or the change of composition of fluid in the part 6a of the patient's body 6 of the patient over a period of time, e.g. based on a comparison between data associated with or representing a plurality of reflection coefficients and/or transmission coefficients, which may be received from the controller 8a, and the data associated with or representing the one or more pre-determined reflection coefficients and/or transmission coefficients, respectively,

The method 100 may comprise alerting a user of the system 2, such as law enforcement, or other official organisation, a doctor, parent, carer or other health care professional, e.g. depending on the determined level of fluid, the change of the level of fluid, the composition of fluid and/or the change of the composition of fluid in the part of the patient's body. The method 100 may comprise contacting an emergency service, e.g. to request an ambulance or doctor, e.g. depending on the determined level of fluid, the change of the level of fluid, the composition of fluid and/or the change of the composition of fluid in the part of the patient's body. For example, as described above, an increase or decrease in the level of fluid in the part of the patient's body, e.g. above or below a threshold, may be indicative of a level of hydration, a state of health, a disease, medical condition and/or a need of the patient, such as the patient's need to use a bathroom. Additionally or alternatively, the composition of fluid and/or a change thereof may be indicative of a state of health, a disease, a medical condition and/or a need of the patient. As such, the method 100 described herein may allow for the prediction of possible oncoming adverse events, such as a fall, incontinence, dehydration, a disease or other medical condition. The determined level of fluid, change of the level of fluid, composition of fluid and/or change of the composition of fluid may be used by a doctor or other health professional to make a diagnosis of the disease or medical condition and/or to assist the patient with their need.

It will be appreciated that determining the level of fluid and/or the composition of fluid in the part of the patient or other subject's body may comprise predicting the level of fluid and/or the composition of fluid in the part of a patient or other subject's body, e.g. based on the data associated with or representing the reflection coefficient and/or or the transmission coefficient and the data associated with or representing the one or more pre-determined reflection coefficients and/or transmission coefficients. For example, the method 100 may comprise using a machine learning model, such as the machine learning model described above, to determine or predict the level of fluid, the change of the level of fluid, the composition of fluid and/or the change of the composition of fluid in the part 6a of the patient's body 6, e.g. based on the data associated with or representing the reflection and/or transmission coefficient. Data associated with or representing the pre-determined reflection coefficient and/or transmission coefficient may be used to train and/or verify the machine learning model, as described above.

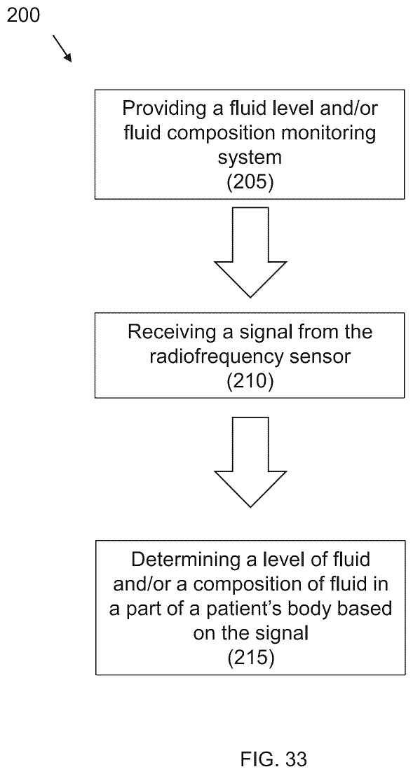

FIG. 33 shows an exemplary flow diagram outlining the steps of an in vivo method of monitoring a level of fluid and/or a composition of fluid. In step 205, the method 200 comprises providing a fluid monitoring system. The fluid level and/or fluid composition monitoring system may include any of the fluid level and/or fluid composition monitoring systems 2 described above. The radiofrequency sensor 4 is included in an item of furnishing or another item that is being used by a patient. The radiofrequency sensor may additionally or alternatively be included in an outdoor or sports item or an outdoor or sports area, as described above. The method 200 is described in relation to a patient. However, it will be appreciated that the term “patient” may be interchangeably be used with the terms “subject” or “person.”

In step 210, the method 200 comprises receiving e.g. by the control system 8, a signal from the radiofrequency sensor 4. In step 215, the method 200 comprises determining, e.g. by the control system 8, a level of fluid and/or a composition of fluid in a part of the patient's body based on the signal.

The signal may comprise a portion of another signal that has been reflected by the part 6a of the patient's body 6 or that has been transmitted through the part 6a of the patient's body 6. The other signal may be or comprise the first signal 10 and the signal may be or comprise the second signal 12.

The method 200 may comprise transmitting the first signal 10 to the part 6a of the patient's body 6. The method 200 may comprise receiving the second signal from the part 6a of the patient's body 6. The first signal 10 may be transmitted by the radiofrequency sensor, the first radiofrequency sensor 4a, one or more radiofrequency sensors 4 of the array 20 of radiofrequency sensors 4 and/or one or more radiofrequency sensors 4a of the first array 20a of radiofrequency sensors 4a, as described above. The second signal 12 may be received by the radiofrequency sensor 4, the second radiofrequency sensor 4b, one or more radiofrequency sensors 4 of the array 20 of radiofrequency sensors 4 and/or one or more radiofrequency sensors 4b of the second array 20b of radiofrequency sensors 4b, as described above.

The method 200 may comprise determining a reflection coefficient and/or a transmission coefficient based on the first and second signals 10, 12.

The method 200 may comprise any of the steps of the method 100 described above in relation to FIG. 29, e.g. to determine the level of fluid and/or composition of fluid based on the determined reflection coefficient or transmission coefficient. One or more steps of the methods may be carried out by the device 8b. The device 8b may be or comprise or be comprised in a mobile phone, smartphone, PDA, tablet computer, laptop computer, and/or the like. The device may comprise the processor 8c, which may be provided in the form of a central processing unit (CPU), maths co-processor (MCP), graphics processing unit (GPU), and/or the like. The processor 8c may be a single core or multicore processor. The device may comprise memory 8d and/or other data storage, which may be implemented on DRAM (dynamic random access memory), SSD (solid state drive), HDD (hard disk drive) or other suitable magnetic, optical and/or electronic memory device. The processor 8c and/or the memory 8d and/or data storage may be arranged locally, e.g. provided in a single device or in multiple devices in in communication at a single location or may be distributed over several local and/or remote devices. The device 8b may comprise a communications module, e.g. a wireless and/or wired communications module. The communications module may be configured to communicate over a cellular communications network, Wi-Fi, Bluetooth, ZigBee, near field communications (NFC), IR, satellite communications, other internet enabling networks and/or the like. The communications module may be configured to communicate via Ethernet or other wired network or connections, via a telecommunications network such as a POTS, PSTN, DSL, ADSL, optical carrier line, and/or ISDN link or network and/or the like, via the cloud and/or via the internet, or other suitable data carrying network. The communications module may be configured to communicate via optical communications such as optical wireless communications (OWC), optical free space communications or Li-Fi or via optical fibres and/or the like. The device 8b may be configured to communicate with the remote server or data store via the communications module.

To provide for interaction with a user, the device 8b may have a screen, e.g., a CRT (cathode ray tube), plasma, LED (light emitting diode) or LCD (liquid crystal display) monitor, for displaying information to the user and an input device, e.g., a keyboard, touch screen, a mouse, a trackball, and the like by which the user can provide input to the computer. Other kinds of devices can be used, for example, feedback provided to the user can be any form of sensory feedback, e.g., visual feedback, auditory feedback, or tactile feedback; and input from the user can be received in any form, including acoustic, speech, or tactile input.

It will be understood that references to a plurality of features may be interchangeably used with references to singular forms of those features, such as for example “at least one” and/or “each”. Singular forms of a feature, such as for example “at least one” or “each,” may be used interchangeably.

It will be appreciated that the terms “first signal” and “other signal” may be interchangeably used. The terms “second signal” and “signal” may be interchangeably used.

It will be appreciated that in the present disclosure, the terms “determining the level of fluid and/or the composition of fluid” may encompass “predicting the level of fluid and/or the composition of fluid”. These terms may be interchangeably used.

Although the disclosure has been described in terms of embodiments as set forth 5 above, it should be understood that these embodiments are illustrative only and that the claims are not limited to those embodiments. Those skilled in the art will be able to make modifications and alternatives in view of the disclosure which are contemplated as falling within the scope of the appended claims. Each feature disclosed or illustrated in the present specification may be incorporated in the disclosure, whether alone or in any 10 appropriate combination with any other feature disclosed or illustrated herein.

1. A fluid level and/or fluid composition monitoring system comprising:
a radiofrequency sensor configured to be included in at least one of:
an item of furnishing or another item for use by a patient or other subject; or
an outdoor or sports item or an outdoor sports area; and
a control system configured to receive a signal from the radiofrequency sensor and to determine a level of fluid and/or a composition of fluid in a part of a patient or other subject's body in vivo based on the signal.

2. The system of claim 1, wherein the radiofrequency sensor is configured to transmit a first signal to the part of the patient or other subject's body and to receive a second signal from the part of the patient or other subject's body.

3. The system of claim 2, wherein the second signal comprises a portion of the first signal that has been reflected by the part of the patient or other subject's body and wherein the signal is or comprises the second signal.

4. The system of claim 2, wherein the system comprises an array of radiofrequency sensors comprising the radiofrequency sensor, one or more radiofrequency sensors of the array of radiofrequency sensors being configured to transmit the first signal to the part of the patient or other subject's body and to receive the second signal from the part of the patient or other subject's body.

5. The system of claim 4, wherein at least one of:
at least one or each radiofrequency sensor of the array of radiofrequency sensors is operable between a first configuration, in which the at least one or each radiofrequency sensor of the array of radiofrequency sensors transmits the first signal or receives the second signal and a second configuration, in which the at least one or each radiofrequency sensor of the array of radiofrequency sensors transmits or receives no signal; and
the array of radiofrequency sensors is configured such that a phase of a first signal transmitted by at least one of the radiofrequency sensors of the array of radiofrequency sensors is shifted relative to a phase of a first signal transmitted by at least one other of the radiofrequency sensors of the array of radiofrequency sensors.

6. The system of claim 1, wherein the radiofrequency sensor is a second radiofrequency sensor and the system comprises a first radiofrequency sensor, the first radiofrequency sensor being configured to transmit a first signal to the part of the patient or other subject's body and the second radiofrequency sensor being configured to receive a second signal from the part of the patient or other subject's body.

7. The system of claim 6, wherein second signal comprises a portion of the first signal that has been transmitted through the part of the patient or other subject's body and wherein the signal is or comprises the second signal.

8. The system of claim 6, wherein the system comprises a first array of radiofrequency sensors comprising the first radiofrequency sensor and a second array of radiofrequency sensors comprising the second radiofrequency sensor, one or more radiofrequency sensors of the first array of radiofrequency sensors being configured to transmit the first signal to the part of the patient or other subject's body and one or more radiofrequency sensors of the second array of radiofrequency sensors being configured to receive the second signal from the part of the patient or other subject's body.

9. The system of claim 7, wherein the first and second radiofrequency sensors and/or the first and second arrays of radiofrequency sensors are configured to be included in one or more items of furnishing or one or more other items such that the first and second radiofrequency sensors and/or the first and second arrays of radiofrequency sensors are on opposite sides of the part of the patient or other subject's body.

10. The system of claim 8, wherein at least one of:
at least one or each radiofrequency sensor of the first array of radiofrequency sensors is operable between a first configuration, in which the at least one or each radiofrequency sensor of the first array of radiofrequency sensors transmits the first signal and a second configuration, in which the at least one or each radiofrequency sensor of the first array of radiofrequency sensors transmits no signal;
the first array of radiofrequency sensors is configured such that a phase of a first signal transmitted by at least one of the radiofrequency sensors of the first array of radiofrequency sensors is shifted relative to a phase of a first signal transmitted by at least one other of the radiofrequency sensors of the first array of radiofrequency sensors.

11. The system of claim 8, wherein at least one or each radiofrequency sensor of the second array of radiofrequency sensors is operable between a first configuration, in which the at least one or each radiofrequency sensor of the second array of radiofrequency sensors receives the second signal and a second configuration, in which the at least one or each radiofrequency sensor of the second array of radiofrequency sensors receives no signal.

12. The system of claim 1, wherein the control system is configured to determine a reflection coefficient and/or a transmission coefficient based on the signal.

13. The system of claim 12, wherein the control system is configured to at least one of:
compare the determined reflection coefficient and/or transmission coefficient to one or more pre-determined reflection coefficients and/or transmission coefficients, respectively, the one or more pre-determined reflection coefficients and/or transmission coefficients being associated with a level of fluid in the part of the patient or other subject's body, a level of fluid in a same part of another patient or subject's body, a composition of fluid in the part of the patient or other subject's body and/or a composition of fluid in the same part of other patient or subject's body; and
determine the level of fluid, a change of the level of fluid, the composition of fluid and/or a change of the composition of fluid in the part of the patient or other subject's body based on the comparison between the determined reflection coefficient and/or transmission coefficient and the one or more pre-determined reflection coefficients and/or transmission coefficients, respectively.

14. The system of claim 13, wherein the control system is configured to determine the level of fluid, the change of the level of fluid the composition of fluid, the change of the composition of fluid in the part of the patient or other subject's body over a period of time based on a comparison between a plurality of determined reflection coefficients and/or transmission coefficients and the one or more pre-determined reflection coefficients or transmissions coefficients, respectively.

15. The system of claim 13, wherein the control system is configured to alert a user of the system, depending on the determined level of fluid, the determined change of the level of fluid, the determined composition of fluid and/or the determined change of the composition of fluid in the part of the patient or other subject's body.

16. The system of claim 1, wherein the control system uses a machine learning model to determine or predict the level of fluid, the change of the level of fluid, the composition of fluid and/or the change of the composition of fluid in the part of the patient or other subject's body.

17. The system of claim 1, wherein the radiofrequency sensor comprises at least one of:
a flexible, bendable or rigid substrate;
a flexible, bendable or rigid conductive element.

18. The system of claim 17, wherein the radiofrequency sensor comprises a first cover portion and a second cover portion, the first and second cover portions being flexible or bendable and configured to encase the flexible, bendable or rigid substrate and the flexible, bendable or rigid conductive element.

19. (canceled)

20. (canceled)

21. The system of claim 1, wherein the part of the patient or other subject's body comprises one or more organs of the patient or other subject, the one or more organs of the patient or other subject comprising at least one of: brain, lungs, kidneys, liver, bladder and heart.

22. An item of furnishing or another item for use by a patient or other subject comprising:
a fluid level and/or fluid composition monitoring system according to claim 1, wherein the radiofrequency sensor is included in the item of furnishing or other item.

23. The item of furnishing or other item of claim 22, wherein the item of furnishing comprises at least one of:
an item of furnishing for use in an outdoor or sports environment;
an item of furniture for the patient or other subject to sleep or rest on;
an item associated with furniture; and
a decorative item; or
wherein the other item comprises at least one of:
an item or part of a vehicle;
an apparatus configured to support and/or restrain a baby or infant in a vehicle;
an item or part of a shelter or dwelling; or
an item of furnishing of the shelter or dwelling.

24. (canceled)

25. (canceled)

26. (canceled)

27. The item of furnishing or other item of claim 22, wherein the radiofrequency sensor is included in the item of furnishing or other item so as to be in proximity to the part of the patient or other subject's body to be monitored.

28. (canceled)

29. An outdoor or sports item or an outdoor or sports area comprising:
a fluid level and/or fluid composition monitoring system according to claim 281, wherein the radiofrequency sensor is included in the outdoor or sports item or the outdoor or sports area.

30. (canceled)

31. (canceled)

32. (canceled)

33. (canceled)

34. (canceled)

35. An in vivo method of monitoring a level of fluid and/or a composition of fluid, the method comprising:
providing a fluid level and/or fluid composition monitoring system according to claim 1, wherein the radiofrequency sensor is included in the item of furnishing or other item, the item of furnishing or other item being used by the patient or other subject, or wherein the radiofrequency sensor is included in the outdoor or sports item or the outdoor or sports area;
receiving a signal from the radiofrequency sensor; and
determining a level of fluid and/or a composition of fluid in the part of the patient or other subject's body based on the signal.
